SEQUENCE LISTING (12 sequences)

SEQ ID NO: 1 - protein, 1068 aa, unidentified
  REGION 1..1068: note = Wild-type PIK3CA Amino Acid Sequence
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 2 - DNA, 3204 bp, unidentified
  misc_feature 1..3204: note = Wild-type PIK3CA Nucleotide Sequence
  source 1..3204: mol_type = unassigned DNA
atgccgccgcgcccgagcagcggcgaactgtggggcattcatctgatgccgccgcgcattctggtggaatgcctgctgccgaacggcatgattgtgaccctggaatgcctgcgcgaagcgaccctgattaccattaaacatgaactgtttaaagaagcgcgcaaatatccgctgcatcagctgctgcaggatgaaagcagctatatttttgtgagcgtgacccaggaagcggaacgcgaagaattttttgatgaaacccgccgcctgtgcgatctgcgcctgtttcagccgtttctgaaagtgattgaaccggtgggcaaccgcgaagaaaaaattctgaaccgcgaaattggctttgcgattggcatgccggtgtgcgaatttgatatggtgaaagatccggaagtgcaggattttcgccgcaacattctgaacgtgtgcaaagaagcggtggatctgcgcgatctgaacagcccgcatagccgcgcgatgtatgtgtatccgccgaacgtggaaagcagcccggaactgccgaaacatatttataacaaactggataaaggccagattattgtggtgatttgggtgattgtgagcccgaacaacgataaacagaaatataccctgaaaattaaccatgattgcgtgccggaacaggtgattgcggaagcgattcgcaaaaaaacccgcagcatgctgctgagcagcgaacagctgaaactgtgcgtgctggaatatcagggcaaatatattctgaaagtgtgcggctgcgatgaatattttctggaaaaatatccgctgagccagtataaatatattcgcagctgcattatgctgggccgcatgccgaacctgatgctgatggcgaaagaaagcctgtatagccagctgccgatggattgctttaccatgccgagctatagccgccgcattagcaccgcgaccccgtatatgaacggcgaaaccagcaccaaaagcctgtgggtgattaacagcgcgctgcgcattaaaattctgtgcgcgacctatgtgaacgtgaacattcgcgatattgataaaatttatgtgcgcaccggcatttatcatggcggcgaaccgctgtgcgataacgtgaacacccagcgcgtgccgtgcagcaacccgcgctggaacgaatggctgaactatgatatttatattccggatctgccgcgcgcggcgcgcctgtgcctgagcatttgcagcgtgaaaggccgcaaaggcgcgaaagaagaacattgcccgctggcgtggggcaacattaacctgtttgattataccgataccctggtgagcggcaaaatggcgctgaacctgtggccggtgccgcatggcctggaagatctgctgaacccgattggcgtgaccggcagcaacccgaacaaagaaaccccgtgcctggaactggaatttgattggtttagcagcgtggtgaaatttccggatatgagcgtgattgaagaacatgcgaactggagcgtgagccgcgaagcgggctttagctatagccatgcgggcctgagcaaccgcctggcgcgcgataacgaactgcgcgaaaacgataaagaacagctgaaagcgattagcacccgcgatccgctgagcgaaattaccgaacaggaaaaagattttctgtggagccatcgccattattgcgtgaccattccggaaattctgccgaaactgctgctgagcgtgaaatggaacagccgcgatgaagtggcgcagatgtattgcctggtgaaagattggccgccgattaaaccggaacaggcgatggaactgctggattgcaactatccggatccgatggtgcgcggctttgcggtgcgctgcctggaaaaatatctgaccgatgataaactgagccagtatctgattcagctggtgcaggtgctgaaatatgaacagtatctggataacctgctggtgcgctttctgctgaaaaaagcgctgaccaaccagcgcattggccatttttttttttggcatctgaaaagcgaaatgcataacaaaaccgtgagccagcgctttggcctgctgctggaaagctattgccgcgcgtgcggcatgtatctgaaacatctgaaccgccaggtggaagcgatggaaaaactgattaacctgaccgatattctgaaacaggaaaaaaaagatgaaacccagaaagtgcagatgaaatttctggtggaacagatgcgccgcccggattttatggatgcgctgcagggctttctgagcccgctgaacccggcgcatcagctgggcaacctgcgcctggaagaatgccgcattatgagcagcgcgaaacgcccgctgtggctgaactgggaaaacccggatattatgagcgaactgctgtttcagaacaacgaaattatttttaaaaacggcgatgatctgcgccaggatatgctgaccctgcagattattcgcattatggaaaacatttggcagaaccagggcctggatctgcgcatgctgccgtatggctgcctgagcattggcgattgcgtgggcctgattgaagtggtgcgcaacagccataccattatgcagattcagtgcaaaggcggcctgaaaggcgcgctgcagtttaacagccataccctgcatcagtggctgaaagataaaaacaaaggcgaaatttatgatgcggcgattgatctgtttacccgcagctgcgcgggctattgcgtggcgacctttattctgggcattggcgatcgccataacagcaacattatggtgaaagatgatggccagctgtttcatattgattttggccattttctggatcataaaaaaaaaaaatttggctataaacgcgaacgcgtgccgtttgtgctgacccaggattttctgattgtgattagcaaaggcgcgcaggaatgcaccaaaacccgcgaatttgaacgctttcaggaaatgtgctataaagcgtatctggcgattcgccagcatgcgaacctgtttattaacctgtttagcatgatgctgggcagcggcatgccggaactgcagagctttgatgatattgcgtatattcgcaaaaccctggcgctggataaaaccgaacaggaagcgctggaatattttatgaaacagatgaacgatgcgcatcatggcggctggaccaccaaaatggattggatttttcataccattaaacagcatgcgctgaac

SEQ ID NO: 3 - protein, 1068 aa, synthetic construct
  REGION 1..1068: note = PIK3CA H1047R Amino Acid Sequence
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDARHGGWTTKMDWIFHTIKQHALN

SEQ ID NO: 4 - DNA, 3207 bp, synthetic construct
  misc_feature 1..3207: note = PIK3CA H1047R Nucleotide Sequence
  source 1..3207: mol_type = other DNA
atgcctccacgaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacgtcatggtggctggacaacaaaaatggattggatcttccacacaattaaacagcatgcattgaactga

SEQ ID NO: 5 - protein, 188 aa, unidentified [sequence omitted]

SEQ ID NO: 6 - DNA, 564 bp, unidentified
  misc_feature 1..564: note = Wild-type KRAS Nucleotide Sequence
  source 1..564: mol_type = unassigned DNA
atgaccgaatataaactggtggtggtgggcgcgggcggcgtgggcaaaagcgcgctgaccattcagctgattcagaaccattttgtggatgaatatgatccgaccattgaagatagctatcgcaaacaggtggtgattgatggcgaaacctgcctgctggatattctggataccgcgggccaggaagaatatagcgcgatgcgcgatcagtatatgcgcaccggcgaaggctttctgtgcgtgtttgcgattaacaacaccaaaagctttgaagatattcatcattatcgcgaacagattaaacgcgtgaaagatagcgaagatgtgccgatggtgctggtgggcaacaaatgcgatctgccgagccgcaccgtggataccaaacaggcgcaggatctggcgcgcagctatggcattccgtttattgaaaccagcgcgaaaacccgccagggcgtggatgatgcgttttataccctggtgcgcgaaattcgcaaacataaagaaaaaatgagcaaagatggcaaaaaaaaaaaaaaaaaaagcaaaaccaaatgcgtgattatg

SEQ ID NO: 7 - protein, 188 aa, synthetic construct
  REGION 1..188: note = KRAS G12V Amino Acid Sequence
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 8 - DNA, 564 bp, synthetic construct
  misc_feature 1..564: note = KRAS G12V Nucleotide Sequence
  source 1..564: mol_type = other DNA
atgaccgaatataaactggtggtggtgggcgcggtggtggtgggcaaaagcgcgctgaccattcagctgattcagaaccattttgtggatgaatatgatccgaccattgaagatagctatcgcaaacaggtggtgattgatggcgaaacctgcctgctggatattctggataccgcgggccaggaagaatatagcgcgatgcgcgatcagtatatgcgcaccggcgaaggctttctgtgcgtgtttgcgattaacaacaccaaaagctttgaagatattcatcattatcgcgaacagattaaacgcgtgaaagatagcgaagatgtgccgatggtgctggtgggcaacaaatgcgatctgccgagccgcaccgtggataccaaacaggcgcaggatctggcgcgcagctatggcattccgtttattgaaaccagcgcgaaaacccgccagggcgtggatgatgcgttttataccctggtgcgcgaaattcgcaaacataaagaaaaaatgagcaaagatggcaaaaaaaaaaaaaaaaaaagcaaaaccaaatgcgtgattatg

SEQ ID NO: 9 - DNA, 48 bp, synthetic construct
  misc_feature 1..48: note = shRNA 1 for p53
  source 1..48: mol_type = other DNA
gaatgaggccttggaattaaactcgagtttaattccaaggcctcattc

SEQ ID NO: 10 - DNA, 47 bp, synthetic construct
  misc_feature 1..47: note = shRNA 2 for p53
  source 1..47: mol_type = other DNA
tggcacctacagtgaaatttctcgagaaatttcactgtaggtgccag

SEQ ID NO: 11 - DNA, 11599 bp, synthetic construct
  misc_feature 1..11599: note = pHAGE-PIK3CA-H1047R Nucleotide Sequence
  source 1..11599: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggactagtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcggccctgcagatatcaacaagtttgtacaaaaaagcaggcaccatgcctccaagaccatcatcaggtgaactgtggggcatccacttgatgcccccaagaatcctagtagaatgtttactaccaaatggaatgatagtgactttagaatgcctccgtgaggctacattaataaccataaagcatgaactatttaaagaagcaagaaaataccccctccatcaacttcttcaagatgaatcttcttacattttcgtaagtgttactcaagaagcagaaagggaagaattttttgatgaaacaagacgactttgtgaccttcggctttttcaaccctttttaaaagtaattgaaccagtaggcaaccgtgaagaaaagatcctcaatcgagaaattggttttgctatcggcatgccagtgtgtgaatttgatatggttaaagatccagaagtacaggacttccgaagaaatattctgaacgtttgtaaagaagctgtggatcttagggacctcaattcacctcatagtagagcaatgtatgtctatcctccaaatgtagaatcttcaccagaattgccaaagcacatatataataaattagataaagggcaaataatagtggtgatctgggtaatagtttctccaaataatgacaagcagaagtatactctgaaaatcaaccatgactgtgtaccagaacaagtaattgctgaagcaatcaggaaaaaaactcgaagtatgttgctatcctctgaacaactaaaactctgtgttttagaatatcagggcaagtatattttaaaagtgtgtggatgtgatgaatacttcctagaaaaatatcctctgagtcagtataagtatataagaagctgtataatgcttgggaggatgcccaatttgatgttgatggctaaagaaagcctttattctcaactgccaatggactgttttacaatgccatcttattccagacgcatttccacagctacaccatatatgaatggagaaacatctacaaaatccctttgggttataaatagtgcactcagaataaaaattctttgtgcaacctacgtgaatgtaaatattcgagacattgataagatctatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaagaaggatgaaacacaaaaggtacagatgaagtttttagttgagcaaatgaggcgaccagatttcatggatgctctacagggctttctgtctcctctaaaccctgctcatcaactaggaaacctcaggcttgaagagtgtcgaattatgtcctctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacgtcatggtggctggacaacaaaaatggattggatcttccacacaattaaacagcatgcattgaactagaacccagctttcttgtacaaagtggttgatatccagcacagtggcggccgctcgagtctagagggcccgcggttcgaaggtaagcctatccctaaccctctcctcggtctcgattctacgcgtaccggttagtaatgagatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacacatatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctaaatcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

SEQ ID NO: 12 - DNA, 9079 bp, synthetic construct
  misc_feature 1..9079: note = pHAGE-KRAS-G12V Nucleotide Sequence
  source 1..9079: mol_type = other DNA
tggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccgaattcacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggactagtcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaagcggccctgcagatatcaacaagtttgtacaaaaaagcaggcaccatgactgaatataaacttgtggtagttggagctgttggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtagaacccagctttcttgtacaaagtggttgatatccagcacagtggcggccgctcgagtctagagggcccgcggttcgaaggtaagcctatccctaaccctctcctcggtctcgattctacgcgtaccggttagtaatgagatccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacacatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaatcgatagatcctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgagatcctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggcccgggttaattaaggaaagggctagatcattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagcaagctcatggctgactaattttttttatttatgcagaggccgaggccgcctcggcctctgagctattccagaagtagtgaggaggcttttttggaggcctaggcttttgcaaaaagctccccgtggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctgtggtttcatttactctaaacctgtgattcctctgaattattttcattttaaagaaattgtatttgttaaatatgtactacaaacttagtagt

(19) United States
(12) Patent Application Publication
(10) Pub. No.: US 2026/0223823 A1
(43) Pub. Date: Aug. 6, 2026

(54) COMPOSITIONS AND METHODS FOR GENERATING PORCINE OVARIAN TUMOR MODEL

(71) Applicant: Georgia Tech Research Corporation, Atlanta, GA (US)

(72) Inventor: Stephen N. Housley, Atlanta, GA (US)

(21) Appl. No.: 19/457,823

(22) Filed: Jan. 23, 2026

Related U.S. Application Data
(60) Provisional application No. 63/749,269, filed on Jan. 24, 2025.

(51) Int. Cl. A01K 67/0278; C12N 9/12; C12N 9/14

(52) U.S. Cl. CPC A01K 67/0278; C12N 9/1205; C12N 9/14; C12Y 207/01153; C12Y 306/05002; A01K 2217/052; A01K 2217/058; A01K 2227/108; A01K 2267/0331

(57) ABSTRACT

Provided herein are orthotopic non-human mammal models of ovarian cancer and methods of generating the non-human mammal models.

### CROSS-REFERENCE TO RELATED APPLICATIONS

This application claims the benefit of U.S. Provisional Patent Application No. 63/749,269, filed Jan. 24, 2025, which is incorporated by reference herein in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on Mar. 27, 2026, is named “GTZ-00301.xml” and is 46,994 bytes in size. The sequence listing contained in this .XML file is part of the specification and is hereby incorporated by reference herein in its entirety.

### BACKGROUND OF THE DISCLOSURE

Ovarian cancer (OC) remains one of the most lethal gynecological malignancies due to its asymptomatic nature and late-stage diagnosis (Morgan, R. D. et al. Lancet Oncol 22, 277-288 (2021); Peres, L. C. et al. J Natl Cancer Inst 111, 60-68 (2019); Fung-Kee-Fung, M. et al. Curr Oncol 14, 195-208 (2007); Hanker, L. C. et al. Annals of Oncology 23, 2605-2612 (2012); Bayraktar, E., Chen, S., Corvigno, S., Liu, J. & Sood, A. K. Cancer Cell 42, 1631-1636 (2024); Prat, J., D'Angelo, E. & Espinosa, I. Hum Pathol 80, 11-27 (2018)). While murine models are ubiquitously used and they have produced tremendous advances in the understanding and treatment of cancer, the fact remains that only a few percent of diagnostic or drug candidates identified in preclinical studies successfully translate to clinical practice (Sharpless, N. E. & Depinho, R. A. Nat Rev Drug Discov 5, 741-754 (2006); Kola, I. & Landis, J. Nat Rev Drug Discov 3, 711-716 (2004); Reichert, J. M. & Wenger, J. B. Drug Discovery Today 13, 30-37 (2008); Singh, M., Murriel, C. L., & Johnson, L. (2012). Cancer research, 72(11), 2695-2700). Murine models may not faithfully mimic human OC and response to interventions, potentially due to interspecies differences in physiology, tumor microenvironment, and genetics. Model inaccuracy may contribute to the low drug approval rate for potential cancer therapeutics and may impede diagnostic or medical device development. These limitation underscores the need for more representative large animal models that can bridge the gap between preclinical studies and human clinical trials.

Porcine models have demonstrated exceptional translational value in the fields of trauma, transplantation, and cardiovascular disease (Swindle, M. M., Makin, A., Herron, A. J., ClubbJr, F. J. & Frazier, K. S. Vet Pathol 49, 344-356 (2012)). Compared to murine models, pigs share more similarity to humans with respect to size, anatomy, physiology, pathophysiological responses, and sequence homology. Pigs could have greater accuracy in cancer modeling than do mice (Schook, L. B. et al. Annu Rev Anim Biosci 3, 219-244 (2015)). Currently, there are no validated large-animal models for OC (Joshi, K. et al. Trends Cancer 10, 182-184 (2024); Perleberg, C., Kind, A. & Schnieke, A. Dis Model Mech 11, dmm030783 (2018)). Although some proof-of-principle studies in pig have been published, they utilized either immunodeficient or sexually immature piglets to grow human OC cells both of which complicate their utility (Boettcher, A. N. et al. Front Oncol 9, 9 (2019)). A porcine OC model would permit research and development of therapeutics, imaging instrumentation, and interventional devices suitable for rapid translation to human OC patients. While the recently developed transgenic ‘Oncopig’ line has successfully modeled solid tumors with organ specificity, they carry only two oncogenic mutations (Schook, L. B. et al. PLoS One 10, e0128864 (2015)). As a result, Oncopigs lack the flexibility to model the molecular diversity observed in human cancer.

### SUMMARY

Provided herein are non-human mammal models of ovarian cancer. In some aspects, an orthotopic non-human mammal model of ovarian cancer comprises ovarian tissue with at least one cancer cell, wherein the ovarian tissue expresses an exogenous mutant PIK3CA polypeptide and an exogenous mutant KRAS polypeptide, and wherein the ovarian tissue has reduced expression of endogenous p53.

In some aspects, an orthotopic non-human mammal model of ovarian cancer comprises ovarian tissue with at least one cancer cell induced by expression of an exogenous mutant PIK3CA polypeptide and an exogenous mutant KRAS polypeptide, and reduced expression of endogenous p53.

Provided herein, are methods of generating a non-human mammal model of ovarian cancer. In some aspects, a method for producing an orthotopic non-human mammal model of ovarian cancer, comprises introducing (i) a first nucleotide sequence encoding an exogenous mutant PIK3CA polypeptide, (ii) a second nucleotide sequence encoding an exogenous mutant KRAS polypeptide, and (iii) at least one inhibitory RNA targeting p53, into an ovarian tissue of a non-human mammal model under conditions effective for the formation ovarian cancer, thereby producing an orthotopic non-human mammal model of ovarian cancer.

In some embodiments, the mammal is porcine.

In some embodiments, the mutant PIK3CA polypeptide comprises an H1047R amino acid substitution. In some embodiments, the mutant PIK3CA polypeptide comprises an amino acid sequence at least 90% identical to SEQ ID NO: 1.

In some embodiments, the mutant KRAS polypeptide comprises a G12V amino acid substitution. In some embodiments, the mutant KRAS polypeptide comprises an amino acid sequence at least 90% identical to SEQ ID NO: 5.

In some embodiments, the ovarian tissue comprises a nucleotide sequence encoding the exogenous mutant PIK3CA polypeptide. In some embodiments, the nucleotide sequence encoding the exogenous mutant PIK3CA polypeptide comprises a sequence at least 90% identical to SEQ ID NO: 2.

In some embodiments, the ovarian tissue comprises a nucleotide sequence encoding the exogenous mutant KRAS polypeptide. In some embodiments, the nucleotide sequence encoding the exogenous mutant KRAS polypeptide comprises a sequence at least 90% identical to SEQ ID NO: 6.

In some embodiments, the expression of p53 is reduced by compared to ovarian tissue of a wild-type mammal.

In some embodiments, the ovarian tissue comprises a tumor.

In some embodiments, a method for generating a non-human mammal model of ovarian cancer comprises introducing a viral vector comprising the first and second nucleotide sequences. In some embodiments, the method comprises introducing a first viral vector comprising the first nucleotide sequence and a second viral vector comprising the second nucleotide sequence.

In some embodiments, the first and second viral vectors each comprise an EFlalpha promoter. In some embodiments, the first viral vector comprises a nucleotide sequence at least 90% identical to SEQ ID NO: 11. In some embodiments, the second viral vector comprises a nucleotide sequence at least 90% identical to SEQ ID NO: 12.

In some embodiments, at least one inhibitory RNA is an siRNA, shRNA, or miRNA. In some embodiments, at least one inhibitory RNA is an shRNA. In some embodiments, the shRNA comprises a nucleotide sequence at least 90% identical to SEQ ID Nos: 9 or 10.

In some embodiments, a method for generating a non-human mammal model of ovarian cancer comprises two inhibitory RNAs, wherein each inhibitory RNA comprises a different region of complementarity to p53.

In some embodiments, one inhibitory RNA comprises a nucleotide sequence at least 90% identical to SEQ ID No: 9 and one inhibitory RNA comprises a nucleotide sequence at least 90% identical to SEQ ID No:10.

In some embodiments, expression of p53 is reduced compared to expression in ovarian tissue not introduced with the inhibitory RNA.

In some embodiments, (i)-(iii) are introduced via unilateral intraovarian injection. In some embodiments, ovarian cancer is formed 1-3 months after introducing (i)-(iii). In some embodiments, formation of ovarian cancer comprises formation of mass forming lesions.

In some embodiments, ovarian cancer comprises tumors comprising diameters for 30-60 mm. In some embodiments formation of ovarian cancer is determined by imaging. In some embodiments, imaging comprises computed tomography (CT). In some embodiments, imaging comprises magnetic resonance imaging (MRI).

In some embodiments, ovarian cancer comprises increased proliferation relative to a noncancerous ovary.

In some embodiments, the orthotopic non-human mammal model of ovarian cancer comprises circulating tumor cells detectable in whole blood.

### BRIEF DESCRIPTION OF THE DRAWINGS

FIG. 1. depicts schematics of the lentivirus vectors used to inoculate pigs. Genes were chosen based on the number of cases in the American Associate for Cancer Research (AACR) Project GENIE.

FIG. 2A depicts a schematic of the study design and timeline.

FIG. 2B depicts a graph showing the change in weight of pigs over a 6-month time period.

FIG. 3 depicts an external view from laparoscopic identification of ovaries (panel A) prior to (panel B) and during injection (n=3) (panel C). Panels D-E depict axial and coronal contrast CT image of matched pig from baseline one month after OC induction indicates contrast enhancing mass lesions at the site of oncogenic lentiviral injection. The dotted white line indicates the largest axial and coronal dimensions in each sequence. Panels F-I depict axial and coronal T1w MRI image of matched pig three months after OC induction indicates expanded contrast enhancing mass lesions at the site of oncogenic lentiviral injection with (panels F-G) and without contrast (panels H-I).

FIG. 4 depicts gross identification of tumor detected ipsilateral to vector injection. Normal ovaries (panel A) were noted contralateral to vector injection and were indistinguishable from ovaries from control animals (panel B). Panel C depicts a low-power view (H&E) of lentiviral injected ovary indicate. Panel D depicts a higher-power view of insets demonstrates sheets of tumor cells with large nuclei and prominent nucleoli and multi-nucleated cells. Tumors demonstrated cellular regions showing hyperchromasia, high mitotic index (yellow arrowheads) (panel E), SET (solid, pseudoendometrioid or transitional) pattern, severe nuclear atypia and significant nuclear pleomorphism (3× variation in size), with large, peculiar, and multinucleated forms (panel F-G).

FIG. 5 depicts representative confocal microscopic image of whole blood collected immediately prior to surgical induction of ovarian cancer (panels A-D). Detected cells (DAPI) were devoid of OC fluorescent reporter mCherry together with timing of collection indicating their presumptive status as circulating immune cells. Blood sample collected at time of CT revealed the presence of mCherry positive circulating cell (presumptive CTC) (panels E-H). Blood sample collected at time of MRI revealed the presence of expanded mCherry positive circulating cells in large cluster (panels I-L). Ascites fluid collected at termination indicated Cherry positive cells (panels M-P).

### DETAILED DESCRIPTION

The present disclosure provides a non-human mammal model of ovarian cancer, along with methods of generating and using the model. In some aspects, the disclosure is based in part on the discovery of the development of a large-animal model of OC by expressing three of the top four oncogenes observed in human OC in sexually mature, immunocompetent female pigs. As existing pig models comprise immunodeficient pigs, sexually immature piglets, or pigs carrying only two oncogenic mutations, it is believed the porcine model of the disclosure will offer more flexibility to model the molecular diversity observed in human cancer.

In some aspects, the disclosure provides an orthotopic non-human mammal model of ovarian cancer. In some aspects, the orthotopic non-human mammal model comprises ovarian tissue expressing mutated oncogenes. In some aspects, the disclosure provides methods for producing orthotopic non-human mammal models of the disclosure. As described herein, it has been demonstrated that an orthotopic non-human mammal model of ovarian cancer is generated by introducing a mutant PIK3A polypeptide, a mutant KRAS polypeptide, and at least one inhibitory RNA targeting p53.

### Orthotopic Ovarian Cancer Model

In some aspects, the disclosure provides orthotopic non-human mammal models of ovarian cancer.

### Definitions

For the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

The term “about” or “approximately” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

The terms “at least,” “less than” or “more than” prior to a number or series of numbers (e.g., “at least two”) is understood to include the number adjacent to the term “at least,” “less than” or “more than,” and all subsequent numbers or integers that could logically be included, as clear from context. When the term “at least,” “less than” or “more than” is present before a series of numbers or a range, it is understood that “at least,” “less than” or “more than” can modify each of the numbers in the series or range.

The term “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein, “orthotopic” refers to occurring in a normal or usual place in the body. Thus, for example, an “orthotopic” model of ovarian cancer refers to ovarian tissue having ovarian cancer cells.

As used herein, “non-human mammal” can be a non-human primate, e.g., an ape or a monkey; and a farm animal, such as an animal selected from the group consisting of cattle, swine; sheep; goats; horses; and donkeys. Accordingly, the non-human mammal can e.g. be a cow, a bull, a bison, a buffalo, a pig, a big-horn sheep, a pony, a mule, a deer, an elk, a lama, and an alpaca. Similarly, the non-human mammal can be a rodent, such as a mouse or rat.

As used herein “endogenous” refers to any material from or produced inside an organism, cell, tissue or system. Thus, for example, an “endogenous” gene is one that is produced and functions in the same body.

As used herein, the term “exogenous” refers to any material introduced from or produced outside an organism, cell, tissue or system. Thus, for example, an “exogenous” gene is one that is produced outside of the body and is introduced into the body.

As used herein, the terms “identity” and “sequence identity” are used interchangeably and refer to when a polynucleotide or polypeptide sequence is aligned to another polynucleotide or polypeptide sequence and the bases or amino acids are the same and in the same relative position.

As used herein, the term “inhibitory RNA” refers to an RNA molecule whose presence within a cell leads to reduced expression of a transcript to which the inhibitory RNA molecule is targeted. The “inhibitory RNA” molecule can be, for example, a miRNA (either endogenous or artificial) or a shRNA, a precursor of a miRNA (i.e. a Pri-miRNA or Pre-miRNA) or shRNA, or a dsRNA that is either transcribed or introduced directly as an isolated nucleic acid, to a cell or subject.

As used herein, the term “mutant” refers to a gene or protein comprising a change to its nucleotide or polypeptide sequence, respectively. The change to the sequence, as used herein, is a “mutation”. Thus, for example, a “mutation” in a gene sequence can result in a “mutant” protein. A “mutation” can be a deletion, substation, or insertion of a nucleotide or amino acid.

As used herein, the term “nucleotide sequence” refers to the succession of bases within DNA or RNA. Thus, for example, a “nucleotide sequence” of a gene can encode or a protein. Furthermore, a “nucleotide sequence” of an shRNA can target and silence a gene. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “tumor” refers to an abnormal mass of tissues that forms in the body. Tumors can be benign (noncancerous) or malignant (cancerous). Unless otherwise specified, the tumors of the non-human mammal model are considered to be malignant.

As used herein, the term “wild-type” refers to the nature, non-mutated form of a gene or protein. Thus, for example, a wild-type protein is one that is the most common, natural-occurring form.

As used herein, a “vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, and the like.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The terms “polypeptide” and “protein” are used interchangeably herein. The term “polypeptide” refers to a protein or peptide that typically contains or consists of at least 20, and preferably at least 30, such as at least 50 amino acids. The term “peptide” refers to an oligomer containing or consisting of at least 2 amino acids to about 19 amino acids.

### Non-Human Mammals

Provided herein are orthotopic non-human mammal models of ovarian cancer. In some embodiments, a non-human mammal can be any non-human mammal, including, but not limited to, laboratory animals, livestock, and species such as murine, rodent, canine, feline, porcine, equine, bovine, ovine, or non-human primate. In some embodiments, the non-human mammal is a small mammal. In some embodiments, the non-human mammal is a large mammal. In some embodiments, non-human mammals can be mice, rats, rabbits, hamsters, guinea pigs, chinchillas, gerbils, dogs, cats, cows, pigs, sheep, goats, and other mammalian species, known in the art. In some embodiments, the non-human mammal is a mouse or a rat. In some embodiments, the non-human mammal is a nonrodent animal. In some embodiments, the non-human mammal is a pig. In some embodiments, the non-human mammal is a minipig. In some embodiments, the minipig is a Göttingen minipig.

### Compositions for Generating a Non-Human Mammal Model

Provided herein, are compositions for generating a non-human mammal model of ovarian cancer. Various compositions are known to those with ordinary skill in the art to generate animal models. In some embodiments, the disclosure provides compositions to express genes encoding exogenous mutant polypeptides in a non-human mammal model. In some embodiments, the disclosure provides compositions to inhibit gene expression in a non-human mammal model.

### Exogenous Mutant Polypeptides

Provided herein, are compositions comprising one or more exogenous mutant polypeptides for generating a non-human mammal model of ovarian cancer. In some embodiments, a composition comprises exogenous mutant polypeptides for modifying gene expression in a non-human mammal model. In some embodiments, the exogenous mutant polypeptides are encoded by oncogenes. In some embodiments, the oncogenes are associated with bone cancer, pancreatic cancer, skin cancer, head and neck cancer, cutaneous or intraorbital malignant melanoma, uterine cancer, ovarian cancer, rectal cancer, anal cancer, and gastric cancer, testicular cancer, uterine cancer, carcinoma of the fallopian tube, endometrial carcinoma, cervical carcinoma, vaginal carcinoma, vulvar carcinoma, Hodgkin's disease, non-Hodgkin's lymphoma, esophageal cancer, small intestine cancer, endocrine cancer, thyroid cancer, parathyroid gland Cancer, adrenal cancer, soft tissue sarcoma, urethral cancer, penile cancer, acute myeloid leukemia, chronic myeloid leukemia, acute lymphoblastic leukemia, chronic or acute leukemia including chronic lymphocytic leukemia, pediatric solid tumors, lymphocytes lymphoma, bladder cancer, cancer of the kidney or ureter, renal pelvis carcinoma, central nervous system (CNS) tumors, primary CNS lymphoma, tumor angiogenesis, spinal tumors, brainstem gliomas, pituitary adenomas, Kaposi's sarcoma, squamous epithelium cancer, squamous cell carcinoma, or T-cell lymphoma. In some embodiments, the oncogenes are associated with breast cancer, lung cancer, liver cancer, colorectal cancer, pancreatic cancer, and ovarian cancer. In some embodiments, the oncogenes are associated with ovarian cancer.

### PIK3CA

In some aspects, the disclosure provides an orthotopic non-human mammal model of ovarian cancer comprising an exogenous mutant Phosphatidylinositol 3-kinase A (PI3KA) polypeptide. PI3KA is implicated in several cellular processes including malignant transformation, growth factor signaling, inflammation, and immunity. PI3KA phosphorylates and activates AKT. Dysregulation of this pathway is frequently found in cancer, specifically ovarian tumors.

In some embodiments, a non-human mammal model comprises an exogenous mutant PI3KA polypeptide. In some embodiments, the exogenous mutant PI3KA polypeptide comprises a mutant P110α subunit. In some embodiments, the mutant P110α subunit is encoded by a mutant phosphatidylinositol-4,5-bisphosphate 3-kinase catalytic subunit alpha (PIK3CA) gene. In some embodiments, the mutant PI3KCA gene comprises at least 1, at least 2, at least 3, at least 4, or at least 5 mutations. In some embodiments, the mutant PI3KCA gene comprises no more than 1, no more than 2, no more than 3, no more than 4, or no more than 4 mutations. In some embodiments, the mutant PI3KCA gene comprises 1, 2, 3, 4, or 5 mutations.

In some embodiments, the mutant PI3KCA gene results in a mutation located at E542, E545, or H1047. In some embodiments, the mutation is E542K. In some embodiments, the mutation is E545K. In some embodiments, the mutation is H1047L. In some embodiments, the mutation is H1047R. In some embodiments, the exogenous mutant PI3KCA polypeptide comprises an E542K mutation, an E545K mutation, and an H1047R mutation. In some embodiments, the exogenous mutant PI3KCA polypeptide comprises an E542K mutation and an E545K mutation. In some embodiments, the exogenous mutant PI3KCA polypeptide comprises an E542K mutation and an H1047R mutation. In some embodiments, the exogenous mutant PI3KCA polypeptide comprises an E545K mutation and an H1047R mutation. In some embodiments, the exogenous mutant PI3KCA polypeptide comprises an E542K mutation. In some embodiments, the exogenous mutant PI3KCA polypeptide comprises an E545K mutation. In some embodiments, the exogenous mutant PI3KCA polypeptide comprises an H1047R mutation.

In some embodiments, the exogenous PIK3CA polypeptide comprises at least 90%, at least 91%, at least 92%, at least 93%, at least 94%, at least 95%, at least 96%, at least 97%, at least 98%, at least 99%, or at least 100% sequence identity to the amino acid sequence set forth in SEQ ID NO: 1. In some embodiments, the PIK3CA polypeptide comprises the amino acid sequence set forth in SEQ ID NO: 1.

In some embodiments, the exogenous PIK3CA polypeptide is encoded by a nucleotide sequence comprising at least 90%, at least 91%, at least 92%, at least 93%, at least 94%, at least 95%, at least 96%, at least 97%, at least 98%, at least 99%, or at least 100% sequence identity to a nucleic acid sequence set forth in SEQ ID NO: 2. In some embodiments, the PIK3CA polypeptide is encoded by a nucleotide sequence set forth in SEQ ID NO: 2.

In some embodiments, the exogenous PIK3CA polypeptide comprises at least 90%, at least 91%, at least 92%, at least 93%, at least 94%, at least 95%, at least 96%, at least 97%, at least 98%, at least 99%, or at least 100% sequence identity to the amino acid sequence set forth in SEQ ID NO: 3. In some embodiments, the PIK3CA polypeptide comprises the amino acid sequence set forth in SEQ ID NO: 3.

In some embodiments, the exogenous PIK3CA polypeptide is encoded by a nucleotide sequence comprising at least 90%, at least 91%, at least 92%, at least 93%, at least 94%, at least 95%, at least 96%, at least 97%, at least 98%, at least 99%, or at least 100% sequence identity to a nucleic acid sequence set forth in SEQ ID NO: 4. In some embodiments, the PIK3CA polypeptide is encoded by a nucleotide sequence set forth in SEQ ID NO: 4.

In some embodiments, the PIK3CA polypeptide comprises the amino acid sequence set forth in Table 1. In some embodiments, the PIK3CA polypeptide is encoded by a nucleotide sequence set forth in Table 1.

In some embodiments, the exogenous mutant PIK3CA gene results in increased activity of PI3KA compared to wild-type protein.

### KRAS

In some aspects, the disclosure provides an orthotopic non-human mammal model of ovarian cancer comprising an exogenous mutant Kirsten rat sarcoma virus (KRAS) polypeptide. KRAS encodes the KRAS GTPase, responsible for activating PI3K in the PI3K-AKT-mTOR pathway. Mutant K-Ras is implicated in one-third of all cancers, including ovarian cancer.

In some embodiments, a non-human mammal model comprises an exogenous mutant KRAS GTPase. In some embodiments, the mutant KRAS GTPase is encoded by a mutant KRAS gene. In some embodiments, the mutant KRAS gene comprises at least 1, at least 2, at least 3, at least 4, or at least 5 mutations. In some embodiments, the mutant KRAS gene comprises no more than 1, no more than 2, no more than 3, no more than 4, or no more than 4 mutations. In some embodiments, the mutant KRAS gene comprises 1, 2, 3, 4, or 5 mutations.

In some embodiments, the KRAS mutation is located at G12, G13, or Q61. In some embodiments, the KRAS mutation is selected from Q61H, Q61R, G13D, G13C, A146T, G12D, G12V, G12R, G12C, or G12A mutation. In some embodiments, the KRAS mutation is a G12C mutation. In some embodiments, the KRAS mutation is a G12D mutation. In some embodiments, the KRAS mutation is a G12V mutation.

In some embodiments, the exogenous KRAS polypeptide comprises at least 90%, at least 91%, at least 92%, at least 93%, at least 94%, at least 95%, at least 96%, at least 97%, at least 98%, at least 99%, or at least 100% sequence identity to the amino acid sequence set forth in SEQ ID NO: 5. In some embodiments, the KRAS polypeptide comprises the amino acid sequence set forth in SEQ ID NO: 5.

In some embodiments, the exogenous KRAS polypeptide is encoded by a nucleotide sequence comprising at least 90%, at least 91%, at least 92%, at least 93%, at least 94%, at least 95%, at least 96%, at least 97%, at least 98%, at least 99%, or at least 100% sequence identity to a nucleic acid sequence set forth in SEQ ID NO: 6. In some embodiments, the KRAS polypeptide is encoded by a nucleotide sequence set forth in SEQ ID NO: 6.

In some embodiments, the exogenous KRAS polypeptide comprises at least 90%, at least 91%, at least 92%, at least 93%, at least 94%, at least 95%, at least 96%, at least 97%, at least 98%, at least 99%, or at least 100% sequence identity to the amino acid sequence set forth in SEQ ID NO: 7. In some embodiments, the KRAS polypeptide comprises the amino acid sequence set forth in SEQ ID NO: 7.

In some embodiments, the exogenous KRAS polypeptide is encoded by a nucleotide sequence comprising at least 90%, at least 91%, at least 92%, at least 93%, at least 94%, at least 95%, at least 96%, at least 97%, at least 98%, at least 99%, or at least 100% sequence identity to a nucleic acid sequence set forth in SEQ ID NO: 8. In some embodiments, the KRAS polypeptide is encoded by a nucleotide sequence set forth in SEQ ID NO: 8.

In some embodiments, the KRAS polypeptide comprises the amino acid sequence set forth in Table 2. In some embodiments, the KRAS polypeptide is encoded by a nucleotide sequence set forth in Table 2.

In some embodiments, the exogenous mutant KRAS gene results in increased activity of KRAS compared to wild-type protein.

### RNA Interference

In some embodiments, the composition for generating an orthotopic non-human mammal model of ovarian cancer comprises an inhibitory RNA. Accordingly, provided herein, are compositions and methods to inhibit gene expression with RNA interference (RNAi). In some embodiments, the RNAi is small interfering RNA (siRNA), a microRNA (miRNA), or a small hairpin RNA (shRNA).

As used herein “shRNA” or “small hairpin RNA” (also called stem loop) is a type of siRNA. In one embodiment, shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow. shRNAs functions as RNAi and/or siRNA species but differs in that shRNA species are double stranded hairpin-like structure for increased stability. These shRNAs, as well as other such agents described herein, can be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety).

shRNAs are produced as single-strand molecules and form stem loop structures. In some embodiments, the single-strand molecule is at least 20, at least 30, at least 40, at least 50, at least 60, at least 70, at least 80, at least 90, or at least 100 nucleotides in length. In some embodiments, the loop is at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25 nucleotides in length. In some embodiments, the stem is 10, at least 15, at least 20, at least 25, at least 30, at least 35, or at least 40 base pairs in length.

In some embodiments the shRNA comprises a 3′ overhang. In some embodiments, the 3′ overhang is at least 1, at least 2, at least 3, at least 4, or at least 5 nucleotides in length. In some embodiments, the 3′ overhang is 2 nucleotides in length.

In some embodiments, the shRNA targets an endogenous gene of the non-human mammal model. In some embodiments, the endogenous gene is an oncogene. In some embodiments, the shRNA targets endogenous p53. In some embodiments, the shRNA comprises a region of complementarity to p53.

In some embodiments, administering shRNA results in a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the RNAi. In one preferred embodiment, the mRNA levels are decreased by at least about 70%, about 80%, about 90%, about 95%, about 99%, about 100%.

In some embodiments, the shRNA comprises a nucleotide sequence at least 90%, at least 91%, at least 92%, at least 93%, at least 94%, at least 95%, at least 96%, at least 97%, at least 98%, at least 99%, at least 100% identical to the nucleotide sequence set forth in SEQ ID NO: 9. In some embodiments, the shRNA comprises a nucleotide sequence at least 90%, at least 91%, at least 92%, at least 93%, at least 94%, at least 95%, at least 96%, at least 97%, at least 98%, at least 99%, at least 100% identical to the nucleotide sequence set forth in SEQ ID NO: 10.

In some embodiments, the shRNA comprises a nucleotide sequence set forth in Table 3.

### Viral Vectors

Provided herein, are compositions comprising viral vectors encoding the exogenous mutant polypeptides and/or inhibitory RNA(s) for generating a non-human mammal model of ovarian cancer. In some embodiments, the composition comprises a vector to deliver exogenous oncogenes. In some embodiments, the composition comprises a vector to deliver an inhibitory RNA. In some embodiments, the vector is a recombinant expression vector.

In some embodiments, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, etc.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193). Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the composition comprises a lentiviral vector. There are several lentiviral vector backbones known to those with ordinary skill in the art. In some embodiments, the vector backbone is at least 500, at least 1000, at least 1500, at least 2000, at least 2500, at least 3000, at least 3500, at least 4000, at least 4500, at least 5000, at least 5500, at least 6000, at least 6500, at least 7000, at least 7500, at least 8000, at least 8500, at least 9000, at least 9500, or at least 10000 base pairs in length. In some embodiments, the vector backbone is at least 3500 base pairs in length. In some embodiments, the vector backbone is at least 4000 base pairs in length. In some embodiments, the vector backbone is at least 4500 base pairs in length. In some embodiments, the vector backbone is at least 5000 base pairs in length. In some embodiments, the vector backbone is at least 5500 base pairs in length. In some embodiments, the vector backbone is at least 6000 base pairs in length. In some embodiments, the vector backbone is at least 6500 base pairs in length.

In some embodiments, the vector backbone is no more than 500, no more than 1000, no more than 1500, no more than 2000, no more than 2500, no more than 3000, no more than 3500, no more than 4000, no more than 4500, no more than 5000, no more than 5500, no more than 6000, no more than 6500, no more than 7000, no more than 7500, no more than 8000, no more than 8500, no more than 9000, no more than 9500, no more than 10000 base pairs in length. In some embodiments, the vector backbone is no more than 3500 base pairs in length. In some embodiments, the vector backbone is no more than 4000 base pairs in length. In some embodiments, the vector backbone is no more than 4500 base pairs in length. In some embodiments, the vector backbone is no more than 5000 base pairs in length. In some embodiments, the vector backbone is no more than 5500 base pairs in length. In some embodiments, the vector backbone is no more than 6000 base pairs in length. In some embodiments, the vector backbone is no more than 6500 base pairs in length.

In some embodiments, the vector backbone is about 1000 to about 2500 base pairs in length. In some embodiments, the vector backbone is about 2500 to about 4000 base pairs in length. In some embodiments, the vector backbone is about 4000 to about 5500 base pairs in length. In some embodiments, the vector backbone is about 5500 to about 7000 base pairs in length.

In some embodiments, the vector backbone is selected from pCDH, pLKO1, pMD2.G, pCMV, pLKO.1 puro, pLVTHM, pLL3.7, among others. In some embodiments, the vector backbone is pCDH. In some embodiments, the vector backbone is pLKO1.

In some embodiments, the lentiviral vector comprises a promoter for the expression of mutant oncogenes. In some embodiments, the promoter for the expression of mutant oncogenes is selected from CMV, EF-1α, H1, U6, PGK, SV40, and MMTV. In some embodiments, the promoter for the expression of mutant oncogenes is EF-1α. In some embodiments, the lentiviral vector comprises promoter for the expression of shRNA targeting a tumor suppressor. In some embodiments, the promoter for the expression of the shRNA is selected from CMV, EF-1α, H1, U6, PGK, SV40, and MMTV. In some embodiments, the promoter for the expression of the shRNA is U6. In some embodiments, the promoter for the expression of the shRNA is H1.

In some embodiments, the lentiviral vector is multicistronic. In some embodiments, the lentiviral vector comprises a first promoter and a second promoter. In some embodiments, the first promoter is the same as the second promoter. In some embodiments, the first promoter is different than the second promoter. In some embodiments, the first promoter is selected from CMV, EF-1α, H1, U6, PGK, SV40, and MMTV and the second promoter is selected from CMV, EF-1α, H1, U6, PGK, SV40, and MMTV. In some embodiments, the first promoter is U6, and the second promoter is selected from CMV, EF-1α, H1, U6, PGK, SV40, and MMTV. In some embodiments, the first promoter is H1, and the second promoter is selected from CMV, EF-1α, H1, U6, PGK, SV40, and MMTV. In some embodiments, the first promoter is selected from CMV, EF-1α, H1, U6, PGK, SV40, and MMTV, and the second promoter is PGK.

In some embodiments, the lentiviral vector comprises from 5′ to 3′, a first promoter, an oncogene, a second promoter, and a reporter gene. In some embodiments, the lentiviral vector comprises from 5′ to 3′, a first promoter, an shRNA, a second promoter, and a reporter gene. In some embodiments, the lentiviral vector comprises an internal ribosomal entry site (IRES). In some embodiments, a lentiviral vector comprises from 5′ to 3′, a promoter, an oncogene, an IRES, and a reporter gene. In some embodiments, a lentiviral vector comprises from 5′ to 3′, a promoter, an shRNA, an IRES, and a reporter gene. In some embodiments, the reporter gene is selected from mCherry, GFP, YFP, RFP, or lacZ, among others. In some embodiments, the reporter gene is mCherry. In some embodiments, the reporter gene is YFP.

In some embodiments, the lentiviral vector is encoded by a nucleotide sequence comprising at least 90%, at least 91%, at least 92%, at least 93%, at least 94%, at least 95%, at least 96%, at least 97%, at least 98%, at least 99%, or at least 100% sequence identity to a nucleic acid sequence set forth in SEQ ID NO: 11. In some embodiments, the lentiviral vector is encoded by a nucleotide sequence set forth in SEQ ID NO: 11.

In some embodiments, the lentiviral vector is encoded by a nucleotide sequence comprising at least 90%, at least 91%, at least 92%, at least 93%, at least 94%, at least 95%, at least 96%, at least 97%, at least 98%, at least 99%, or at least 100% sequence identity to a nucleic acid sequence set forth in SEQ ID NO: 12. In some embodiments, the lentiviral vector is encoded by a nucleotide sequence set forth in SEQ ID NO: 12.

In some embodiments, the lentiviral vector comprises a nucleotide sequence set forth in Table 4.

### Characteristics of Model

Provided herein, is an orthotopic non-human mammal model of ovarian cancer, wherein the cancer is induced after delivery of oncogenes. In some embodiments, the non-human mammal model comprises at least one cancer cell in ovarian tissue. In some embodiments, the ovarian tissue expresses exogenous mutant PIK3A. In some embodiments, the ovarian tissue expresses exogenous mutant KRAS. In some embodiments, the ovarian tissue comprises reduced expression of endogenous p53. Methods of visualizing and quantifying gene expression are known to those with ordinary skill in the art. In some embodiments, methods for visualizing and quantifying gene expression include, but are not limited to, quantitative polymerase chain reaction (qPCR), reverse-transcription PCR (RT-PCR), microarray analysis, and Northern Blot. Methods of visualizing and quantifying protein expression are known to those with ordinary skill in the art. In some embodiments, methods for visualizing and quantifying protein expression include, but are not limited to, flow cytometry, Western Blot, mass spectrometry, enzyme-linked immunosorbent assay (ELISA), Bradford assay, Bicinchoninic acid assay, immunohistochemistry, and immunofluorescent imaging.

In some embodiments, oncogene expression is measured with immunofluorescent imaging. In some embodiments, oncogene expression is measured with immunohistochemistry.

In some aspects, the non-human mammal model of ovarian cancer comprises ovarian tumors or mass forming lesions. In some embodiments, the non-human mammal model comprises mass forming lesions with perilesional edema at the carcinoma site. Examples of lesions include, but are not limited to, wounds, ulcers, abscesses, sores, cysts and tumors.

Methods of visualizing and measuring lesions and tumors are known to those with ordinary skill in the art. In some embodiments, Hematoxylin ad Eosin (H&E) standing is used to visualize lesions and tumors. In some embodiments, magnetic resonance imaging (MRI) is used to visualize lesions and tumors. In some embodiments, computed tomography (CT) is used to visualize lesions and tumors.

In some embodiments, tumor size is evaluated by CT. In some embodiments, tumor size is evaluated by MRI.

In some embodiments, the ovarian tumor size ranges from 10 to 20 mm in diameter. In some embodiments, the ovarian tumor size ranges from 20 to 30 mm in diameter. In some embodiments, the ovarian tumor size ranges from 30 to 40 mm in diameter. In some embodiments, the ovarian tumor size ranges from 40 to 50 mm in diameter. In some embodiments, the ovarian tumor size ranges from 50 to 60 mm in diameter. In some embodiments, the ovarian tumor size ranges from 60 to 70 mm in diameter. In some embodiments, the ovarian tumor size ranges from 70 to 80 mm in diameter. In some embodiments, the ovarian tumor size ranges from 80 to 90 mm in diameter. In some embodiments, the ovarian tumor size ranges from 90 to 100 mm in diameter.

In some embodiments, the tumor comprises cellular regions with hyperchromasia. Hyperchromasia describes cells with dark nuclei. In some embodiments the tumor demonstrates a solid, pseudo endometrioid pattern. In some embodiments the tumor demonstrates a transitional pattern. In some embodiments, cell nuclei exhibit pleomorphism. In some embodiments, cell nuclei exhibit increased size. In some embodiments, cell nuclei exhibit a high mitotic index.

A hallmark of ovarian cancer is the presence of circulating tumor cells (CTCs). In some embodiments, the non-human mammal model of the disclosure exhibits CTCs. In some embodiments, whole blood is filtered to obtain CTCs. In some embodiments, a microwell filter is used to filter whole blood. In some embodiments, filter comprises a diagonal aperture of at least 2, at least 4, at least 6, at least 8, at least 10, or at least 12 m. In some embodiments, the diagonal aperture is between about 8 and about 10 m. In some embodiments, the diagonal aperture is between about 10 and about 12 m. In some embodiments, CTCs are quantified with fluorescent reporters. In some embodiments, the presence of CTCs correlates with detection of mass forming lesions.

Ovarian cancer typically remains covert despite advanced tumorigenesis detectable by clinical imaging. In some embodiments, the non-human mammal model is observed for symptoms after cancer induction. Symptoms can include, but are not limited to, weight loss, muscle loss, changes in bowel and bladder function, and neurologic defects. In some embodiments, the non-human mammal model is asymptomatic after cancer induction. In some embodiments, the non-human mammal model remains alert after cancer induction. In some embodiments, the non-human mammal model remains responsive after cancer induction. In some embodiments, the non-human mammal model maintains weight after cancer induction. In some embodiments, the non-human mammal model maintains muscle after cancer induction. In some embodiments, the non-human mammal model maintains bowel and bladder function after cancer induction.

### Methods for Generating Model

Provided herein, are methods for generating an orthotopic non-human mammal model of ovarian cancer. In some embodiments, the method comprises introducing into an ovarian tissue of a non-human mammal model i) a first nucleotide sequence encoding an exogenous mutant PIK3CA polypeptide, ii) a second nucleotide sequence encoding an exogenous mutant KRAS polypeptide, and iii) at least one inhibitory RNA targeting p53.

In some embodiments, the method comprises introducing i) a first nucleotide sequence encoding an exogenous mutant PIK3CA polypeptide comprising an H1047R mutation, ii) a second nucleotide sequence encoding an exogenous mutant KRAS polypeptide, and iii) at least one inhibitory RNA targeting p53.

In some embodiments, the method comprises introducing i) a first nucleotide sequence encoding an exogenous mutant PIK3CA polypeptide, ii) a second nucleotide sequence encoding an exogenous mutant KRAS polypeptide comprising a G12V mutation, and iii) at least one inhibitory RNA targeting p53.

In some embodiments, the method comprises introducing i) a first nucleotide sequence encoding an exogenous mutant PIK3CA polypeptide, ii) a second nucleotide sequence encoding an exogenous mutant KRAS polypeptide, and iii) at least one inhibitory RNA targeting p53, wherein the inhibitory RNA is an shRNA.

In some embodiments, the method comprises introducing i) a first nucleotide sequence encoding an exogenous mutant PIK3CA polypeptide comprising an H1047R mutation, ii) a second nucleotide sequence encoding an exogenous mutant KRAS polypeptide comprising a G12V mutation, and iii) at least one inhibitory RNA targeting p53.

In some embodiments, the method comprises introducing i) a first nucleotide sequence encoding an exogenous mutant PIK3CA polypeptide comprising an H1047R mutation, ii) a second nucleotide sequence encoding an exogenous mutant KRAS polypeptide, and iii) at least one inhibitory RNA targeting p53, wherein the inhibitory RNA is an shRNA.

In some embodiments, the method comprises introducing i) a first nucleotide sequence encoding an exogenous mutant PIK3CA polypeptide, ii) a second nucleotide sequence encoding an exogenous mutant KRAS polypeptide comprising a G12V mutation, and iii) at least one inhibitory RNA targeting p53, wherein the inhibitory RNA is an shRNA.

In some embodiments, the method comprises introducing i) a first nucleotide sequence encoding an exogenous mutant PIK3CA polypeptide comprising an H1047R mutation, ii) a second nucleotide sequence encoding an exogenous mutant KRAS polypeptide comprising a G12V mutation, and iii) at least one inhibitory RNA targeting p53, wherein the inhibitory RNA is an shRNA.

In some embodiments, the method comprises introducing into an ovarian tissue of a non-human mammal model i) a first nucleotide sequence set forth in SEQ ID NO: 4 encoding an exogenous mutant PIK3CA polypeptide, ii) a second nucleotide sequence encoding an exogenous mutant KRAS polypeptide, and iii) at least one inhibitory RNA targeting p53.

In some embodiments, the method comprises introducing into an ovarian tissue of a non-human mammal model i) a first nucleotide sequence encoding an exogenous mutant PIK3CA polypeptide, ii) a second nucleotide sequence set forth in SEQ ID NO: 8 encoding an exogenous mutant KRAS polypeptide, and iii) at least one inhibitory RNA targeting p53.

In some embodiments, the method comprises introducing into an ovarian tissue of a non-human mammal model i) a first nucleotide sequence encoding an exogenous mutant PIK3CA polypeptide, ii) a second nucleotide sequence encoding an exogenous mutant KRAS polypeptide, and iii) at least one inhibitory RNA targeting p53 set forth in SEQ ID NOs: 9 or 10.

In some embodiments, the method comprises introducing into an ovarian tissue of a non-human mammal model i) a first nucleotide sequence set forth in SEQ ID NO: 4 encoding an exogenous mutant PIK3CA polypeptide, ii) a second nucleotide sequence set forth SEQ ID NO: 8 encoding an exogenous mutant KRAS polypeptide, and iii) at least one inhibitory RNA targeting p53.

In some embodiments, the method comprises introducing into an ovarian tissue of a non-human mammal model i) a first nucleotide sequence set forth in SEQ ID NO: 4 encoding an exogenous mutant PIK3CA polypeptide, ii) a second nucleotide sequence encoding an exogenous mutant KRAS polypeptide, and iii) at least one inhibitory RNA targeting p53 set forth in SEQ ID NOs: 9 or 10.

In some embodiments, the method comprises introducing into an ovarian tissue of a non-human mammal model i) a first nucleotide sequence encoding an exogenous mutant PIK3CA polypeptide, ii) a second nucleotide sequence set forth in SEQ ID NO: 8 encoding an exogenous mutant KRAS polypeptide, and iii) at least one inhibitory RNA targeting p53 set forth in SEQ ID NOs: 9 or 10.

In some embodiments, the method comprises introducing into an ovarian tissue of a non-human mammal model i) a first nucleotide sequence set forth in SEQ ID NO: 4 encoding an exogenous mutant PIK3CA polypeptide, ii) a second nucleotide sequence set forth in SEQ ID NO: 8 encoding an exogenous mutant KRAS polypeptide, and iii) at least one inhibitory RNA targeting p53 set forth in SEQ ID NOs: 9 or 10. In some embodiments, the first and second nucleotide sequences are on the same viral vector. In some embodiments, the first and second nucleotide sequences are on different viral vectors.

Methods of administering vectors to non-human mammal models are known to those with ordinary skill in the art. Methods can include, but are not limited to, viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et., al Adv Drug Deliv Rev. 2012 Sep. 13. pii: 50169-409X(12)00283-9. doi: 10.1016/j.addr.2012.09.023), and the like. In some embodiments, the vectors are introduced to ovarian tissue of a non-human mammal via unilateral intraovarian injection.

In some embodiments, ovarian cancer is formed 1-3 months after cancer induction. In some embodiments, ovarian cancer is formed 3-6 months after cancer induction. In some embodiments, ovarian cancer is formed 6-9 months after cancer induction. In some embodiments, ovarian cancer is formed 9-12 months after cancer induction. In some embodiments, ovarian cancer is formed at least 1 month after cancer induction. In some embodiments, ovarian cancer is formed at least 2 months after cancer induction. In some embodiments, ovarian cancer is formed at least 3 months after cancer induction. In some embodiments, ovarian cancer is formed no more than 1 month after cancer induction. In some embodiments, ovarian cancer is formed no more than 2 months after cancer induction. In some embodiments, ovarian cancer is formed no more than 3 months after cancer induction.

### Methods of Use

Provided herein, are non-human mammal models of ovarian cancer. In some embodiments, the non-human mammal model is used as a model for identifying potential therapeutic compounds, testing the efficacy and/or dosage of therapeutics, and/or pharmacokinetic analyses in cancer (alone or in the presence of co-morbidities). In some embodiments, the non-human mammal model is used as a model for testing medical devices or procedures. In some embodiments, the non-human mammal model is used as a model for screening therapeutic compounds. In some embodiments, the non-human mammal model is used as a pre-clinical toxicology model. In some embodiments, the non-human mammal model is used as a model for identifying biomarkers (e.g., for diagnostic and/or prognostic tests). In some embodiments, the non-human mammal model is used as a personalized tumor model.

### EXAMPLES

### Example 1: Delivery of Top Ovarian Cancer Oncogenes to Immunocompetent, Sexually Mature Pigs

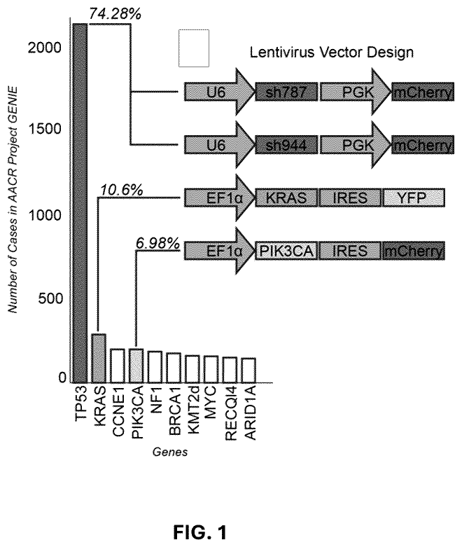

To induce large animal ovarian cancer (OC), a combination of third-generation replication deficient lentiviral vectors was used (FIG. 1). Genes were chosen based on the number of cases in the American Associate for Cancer Research (AACR) Project GENIE. A first vector comprised Pik3ca (H1047R) on a pCDH transfer plasmid backbone under an Ef1α promoter. A second vector comprised KRAS (G12V) on a pCDH transfer plasmid backbone under an Ef1α promoter. A third vector comprised a sequence of shRNA 787 (sh787) targeting p53 on a pLKO1 backbone under the H1 or U6 promoter. A fourth vector comprised a sequence of shRNA 944 (sh944) targeting p53 on a pLKO1 backbone under the H1 or U6 promoter. The vectors were titered at >109IU·mL−1. All vectors were thawed and combined in equal parts (1:1:1:1) immediately prior to inoculation.

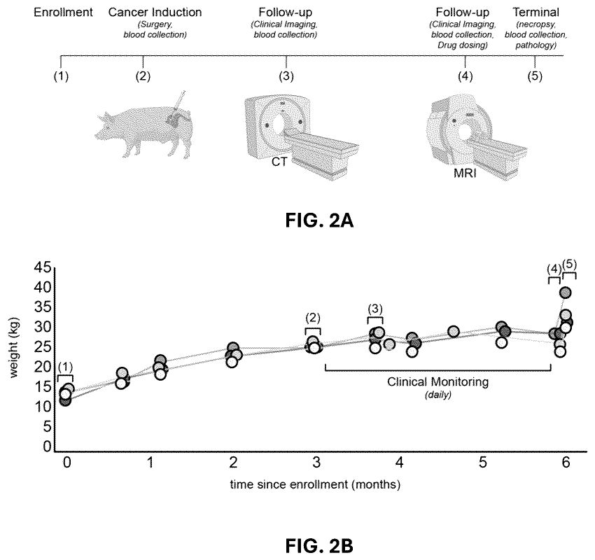

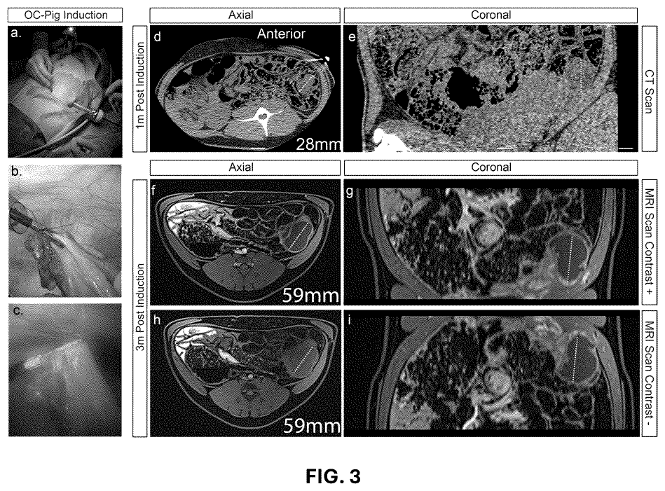

To induce cancer, unilateral intraovarian injections (50 μL) of a lentiviral vector cocktail were performed in sexually mature, immunocompetent, treatment naïve female Gottingen pigs (25.75±1.25 kg, n=4) (FIG. 2A). Change in weight over the course of the study is shown in FIG. 2B. Ovaries were identified either laparoscopically prior to injection (n=3) or after externalization through a small (~7 cm) horizontal incision in the inferior abdominal wall (FIG. 3, panels A-C) as the ovary could not be identified and manipulated to successfully perform a percutaneous injection. Blood collected after surgery revealed no changes in blood serum chemistry relative to pre-operative levels indicating minimal systemic response to surgery (data not shown).

### Example 2: CT and MR Imaging Detect Tumorigenesis Despite Asymptomatic Clinical Presentation

After recovery from cancer induction and surgery as described in Example 1, animals were evaluated daily for clinical deficits for three months (planned endpoint). Computed tomography (CT) imaging was performed at approximately one month after cancer induction, and magnetic resonance imaging (MRI) at endpoint (three months after cancer induction) and whole blood was collected at each imaging session. All animals remained bright, alert, and responsive, with no signs of weight loss, cachexia, neurologic symptoms or changes bowel and bladder function throughout the study. Four-five weeks after intraovarian injection, CT imaging revealed contrast enhancing mass lesions at the site of oncogenic lentiviral injection in three of four animals (FIG. 3, panels D-E), suggestive of successful ovarian cancer induction. At endpoint, three months after cancer induction, animals underwent MRI prior to euthanasia and necropsy. MRI demonstrated T1-weighted (T1w) contrast-enhancing mass forming lesions with marked peri-lesional edema at the site of oncogenic lentiviral injection in all animals (FIG. 3, panels F-I). These radiologic features were consistent with ovarian carcinoma and included maximal tumor diameters ranging from 30-59 mm (n=4). Given all animals remained asymptomatic throughout the study, this collective presentation is consistent with human ovarian cancer which frequently remains covert despite advanced tumorigenesis detectable by clinical imaging.

### Example 3: Histopathological and Immunohistochemical Characterization

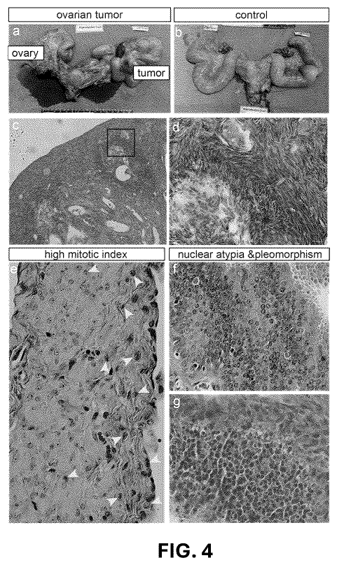

86-89 days after cancer induction as described in Example 1, all animals underwent planned euthanasia and necropsy 1-2 days post MRI after uncomplicated courses. Samples of abdominal fluid were collected for further analysis. Gross evidence of tumors was detected ipsilateral to vector injection. Normal ovaries were noted contralateral to vector injection (FIG. 4 panel A) and were indistinguishable from ovaries from control animals (FIG. 4 panel B). Hematoxylin and Eosin (H&E) staining of slices ipsilateral to the viral vector delivery demonstrated abundant tumor cells in all subjects (FIG. 4 panel C). At low magnification, the tumors demonstrated cellular regions showing hyperchromasia and solid, pseudo endometrioid (SET) or transitional pattern (FIG. 4 panels C-D). Histopathologic features at high magnification included severe nuclear atypia and significant nuclear pleomorphism (3× variation in size), with large, peculiar, and multinucleated forms. Prominent nucleoli were seen, with a high mitotic index (>12 mitotic figures per 10 high power fields). Identified histopathologic features were consistent with those observe in human ovarian carcinomas (FIG. 4 panels E-G). Histopathologic features of ovaries contralateral to injection site (control) revealed grossly normal medulla and cortex containing developing follicles. Normal corpus luteum and ovarian surface epithelium composed of a single cell lining were also noted throughout (data not shown).

Sections sequential to those studied with H&E were stained with anti-mCherry antibodies to directly detect virally transfected oncogenes. Strong mCherry expression was detected throughout tumor sections and was not detectable above background in control ovaries (data not shown). Sections sequential to those studied with H&E were stained with anti-Ki-67 antibodies and demonstrated strong immunopositivity with mean proliferative index of 70% which corresponded with human ovarian cancer sections and was significantly elevated relative to that observed in control ovaries 3% (data not shown).

### Example 4: Circulating Tumor Cells Detection Coincides with Tumorigenesis

An emerging clinical hallmark of ovarian cancer is the presence of circulating tumor cells (CTCs). The presence of CTCs correlates with tumor stage, disease recurrence, and survival and has the potential to act as a liquid biopsy for drug treatment monitoring or as a companion tool to aid in diagnosis. It was tested whether CTCs could be detected from whole blood samples over the course of the three-month study. Using the mCherry fluorescent reporter that was co-transfected with each vector cocktail, the presence or absence of CTCs relative to other circulating cell populations based on their mCherry expression was assessed. Whole blood was passed through a custom microwell filter with diagonal aperture of ~11 μm. This enabled large diameter circulating cells and cell clusters to be retained while smaller cell populations were depleted. While not specific for cancer cells, this form of size exclusion filtering enriches each sample for large diameter populations and enables high-throughput- and confocal-microscopy to image rare circulating cell types that might be lost to cytometry-based techniques. For consistency, ~2 ml of whole blood was tested at each time point to mirror the volume collected during a typical clinical blood draw.

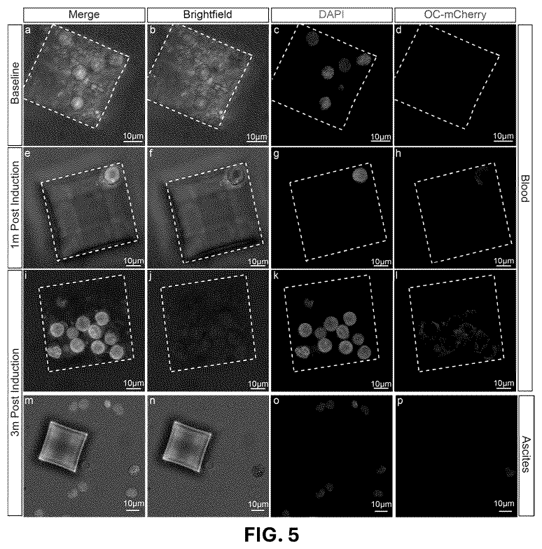

Analysis of whole blood collected at the time of surgery revealed numerous circulating cells were retained on the filter, yet no mCherry expression was detected (FIG. 5, panels A-D). Confirmatory analysis was performed by fixing and staining filters with anti-mCherry antibodies followed by secondary antibody detection. Follow up imaging confirmed the expected absence of virally transfected cells and indicates that the collected cells were likely immune cells. Blood collected at the time of CT imaging (approximately 1 month later than the baseline) was evaluated. As shown in FIG. 5, panels E-H, sparse mCherry expressing cells were detected in each sample. These presumptive CTCs coincided with the detection of contrast enhancing mass lesions on CT. A larger number of mCherry expressing cells was detected in blood collected at end point (approximately 3 months after the baseline) (FIG. 5, panels I-L), possibly indicating the presence of CTC clusters due to their close association. This apparent increase in CTC number was notable as it coincided with the MRI indicating increased tumor volume (FIG. 3). mCherry positive, presumptive ovarian cancer cells were also detected in the ascites fluid collected at necropsy (FIG. 5, panels M-P). These data indicate that the large animal model recapitulates another key feature of human ovarian cancer.

Taken together, the clinical imaging, gross anatomical and histopathological tumor characteristics, and evidence of hematogenous and transcoelomic cancer cell dissemination help to credential the large animal model of ovarian cancer, herein referred to as OC-Pigs. Without wishing to be bound by theory, these data show the OC-Pigs are useful as a translational model for drug and/or diagnostic tool development.

### Sequences

TABLE 1
PIK3CA Sequences

| | | SEQ |
|---|---|---|
| | | ID |
| Description | Sequence | NO |
| Wild-type | MPPRPSSGELWGIHLMPPRILVECLLPNGMIVTLECLREATLITIKHELFK | 1 |
| PIK3CA | EARKYPLHQLLQDESSYIFVSVTQEAEREEFFDETRRLCDLRLFQPFLKV | |
| Amino Acid | IEPVGNREEKILNREIGFAIGMPVCEFDMVKDPEVQDFRRNILNVCKEAV | |
| Sequence | DLRDLNSPHSRAMYVYPPNVESSPELPKHIYNKLDKGQIIVVIWVIVSPN | |
| | NDKQKYTLKINHDCVPEQVIAEAIRKKTRSMLLSSEQLKLCVLEYQGK | |
| | YILKVCGCDEYFLEKYPLSQYKYIRSCIMLGRMPNLMLMAKESLYSQLP | |
| | MDCFTMPSYSRRISTATPYMNGETSTKSLWVINSALRIKILCATYVNVNI | |
| | RDIDKIYVRTGIYHGGEPLCDNVNTQRVPCSNPRWNEWLNYDIYIPDLP | |
| | RAARLCLSICSVKGRKGAKEEHCPLAWGNINLFDYTDTLVSGKMALNL | |
| | WPVPHGLEDLLNPIGVTGSNPNKETPCLELEFDWFSSVVKFPDMSVIEE | |
| | HANWSVSREAGFSYSHAGLSNRLARDNELRENDKEQLKAISTRDPLSEI | |
| | TEQEKDFLWSHRHYCVTIPEILPKLLLSVKWNSRDEVAQMYCLVKDWP | |
| | PIKPEQAMELLDCNYPDPMVRGFAVRCLEKYLTDDKLSQYLIQLVQVLK | |
| | YEQYLDNLLVRFLLKKALTNQRIGHFFFWHLKSEMHNKTVSQRFGLLL | |
| | ESYCRACGMYLKHLNRQVEAMEKLINLTDILKQEKKDETQKVQMKFL | |
| | VEQMRRPDFMDALQGFLSPLNPAHQLGNLRLEECRIMSSAKRPLWLNW | |
| | ENPDIMSELLFQNNEIIFKNGDDLRQDMLTLQIIRIMENIWQNQGLDLRM | |
| | LPYGCLSIGDCVGLIEVVRNSHTIMQIQCKGGLKGALQFNSHTLHQWLK | |
| | DKNKGEIYDAAIDLFTRSCAGYCVATFILGIGDRHNSNIMVKDDGQLFHI | |
| | DFGHFLDHKKKKFGYKRERVPFVLTQDFLIVISKGAQECTKTREFERFQ | |
| | EMCYKAYLAIRQHANLFINLFSMMLGSGMPELQSFDDIAYIRKTLALDK | |
| | TEQEALEYFMKQMNDAHHGGWTTKMDWIFHTIKQHALN | |
| Wild-type | ATGCCGCCGCGCCCGAGCAGCGGCGAACTGTGGGGCATTCATCTGAT | 2 |
| PIK3CA | GCCGCCGCGCATTCTGGTGGAATGCCTGCTGCCGAACGGCATGATTG | |
| Nucleotide | TGACCCTGGAATGCCTGCGCGAAGCGACCCTGATTACCATTAAACAT | |
| Sequence | GAACTGTTTAAAGAAGCGCGCAAATATCCGCTGCATCAGCTGCTGCA | |
| | GGATGAAAGCAGCTATATTTTTGTGAGCGTGACCCAGGAAGCGGAAC | |
| | GCGAAGAATTTTTTGATGAAACCCGCCGCCTGTGCGATCTGCGCCTG | |
| | TTTCAGCCGTTTCTGAAAGTGATTGAACCGGTGGGCAACCGCGAAGA | |
| | AAAAATTCTGAACCGCGAAATTGGCTTTGCGATTGGCATGCCGGTGT | |
| | GCGAATTTGATATGGTGAAAGATCCGGAAGTGCAGGATTTTCGCCGC | |
| | AACATTCTGAACGTGTGCAAAGAAGCGGTGGATCTGCGCGATCTGAA | |
| | CAGCCCGCATAGCCGCGCGATGTATGTGTATCCGCCGAACGTGGAAA | |
| | GCAGCCCGGAACTGCCGAAACATATTTATAACAAACTGGATAAAGGC | |
| | CAGATTATTGTGGTGATTTGGGTGATTGTGAGCCCGAACAACGATAAA | |
| | CAGAAATATACCCTGAAAATTAACCATGATTGCGTGCCGGAACAGGT | |
| | GATTGCGGAAGCGATTCGCAAAAAAACCCGCAGCATGCTGCTGAGC | |
| | AGCGAACAGCTGAAACTGTGCGTGCTGGAATATCAGGGCAAATATAT | |
| | TCTGAAAGTGTGCGGCTGCGATGAATATTTTCTGGAAAAATATCCGCT | |
| | GAGCCAGTATAAATATATTCGCAGCTGCATTATGCTGGGCCGCATGCC | |
| | GAACCTGATGCTGATGGCGAAAGAAAGCCTGTATAGCCAGCTGCCGA | |
| | TGGATTGCTTTACCATGCCGAGCTATAGCCGCCGCATTAGCACCGCGA | |
| | CCCCGTATATGAACGGCGAAACCAGCACCAAAAGCCTGTGGGTGATT | |
| | AACAGCGCGCTGCGCATTAAAATTCTGTGCGCGACCTATGTGAACGT | |
| | GAACATTCGCGATATTGATAAAATTTATGTGCGCACCGGCATTTATCAT | |
| | GGCGGCGAACCGCTGTGCGATAACGTGAACACCCAGCGCGTGCCGT | |
| | GCAGCAACCCGCGCTGGAACGAATGGCTGAACTATGATATTTATATTC | |
| | CGGATCTGCCGCGCGCGGCGCGCCTGTGCCTGAGCATTTGCAGCGTG | |
| | AAAGGCCGCAAAGGCGCGAAAGAAGAACATTGCCCGCTGGCGTGGG | |
| | GCAACATTAACCTGTTTGATTATACCGATACCCTGGTGAGCGGCAAA | |
| | ATGGCGCTGAACCTGTGGCCGGTGCCGCATGGCCTGGAAGATCTGCT | |
| | GAACCCGATTGGCGTGACCGGCAGCAACCCGAACAAAGAAACCCCG | |
| | TGCCTGGAACTGGAATTTGATTGGTTTAGCAGCGTGGTGAAATTTCCG | |
| | GATATGAGCGTGATTGAAGAACATGCGAACTGGAGCGTGAGCCGCGA | |
| | AGCGGGCTTTAGCTATAGCCATGCGGGCCTGAGCAACCGCCTGGCGC | |
| | GCGATAACGAACTGCGCGAAAACGATAAAGAACAGCTGAAAGCGAT | |
| | TAGCACCCGCGATCCGCTGAGCGAAATTACCGAACAGGAAAAAGATT | |
| | TTCTGTGGAGCCATCGCCATTATTGCGTGACCATTCCGGAAATTCTGC | |
| | CGAAACTGCTGCTGAGCGTGAAATGGAACAGCCGCGATGAAGTGGC | |
| | GCAGATGTATTGCCTGGTGAAAGATTGGCCGCCGATTAAACCGGAAC | |
| | AGGCGATGGAACTGCTGGATTGCAACTATCCGGATCCGATGGTGCGC | |
| | GGCTTTGCGGTGCGCTGCCTGGAAAAATATCTGACCGATGATAAACT | |
| | GAGCCAGTATCTGATTCAGCTGGTGCAGGTGCTGAAATATGAACAGT | |
| | ATCTGGATAACCTGCTGGTGCGCTTTCTGCTGAAAAAAGCGCTGACC | |
| | AACCAGCGCATTGGCCATTTTTTTTTTTGGCATCTGAAAAGCGAAATG | |
| | CATAACAAAACCGTGAGCCAGCGCTTTGGCCTGCTGCTGGAAAGCTA | |
| | TTGCCGCGCGTGCGGCATGTATCTGAAACATCTGAACCGCCAGGTGG | |
| | AAGCGATGGAAAAACTGATTAACCTGACCGATATTCTGAAACAGGAA | |
| | AAAAAAGATGAAACCCAGAAAGTGCAGATGAAATTTCTGGTGGAAC | |
| | AGATGCGCCGCCCGGATTTTATGGATGCGCTGCAGGGCTTTCTGAGCC | |
| | CGCTGAACCCGGCGCATCAGCTGGGCAACCTGCGCCTGGAAGAATG | |
| | CCGCATTATGAGCAGCGCGAAACGCCCGCTGTGGCTGAACTGGGAAA | |
| | ACCCGGATATTATGAGCGAACTGCTGTTTCAGAACAACGAAATTATT | |
| | TTTAAAAACGGCGATGATCTGCGCCAGGATATGCTGACCCTGCAGATT | |
| | ATTCGCATTATGGAAAACATTTGGCAGAACCAGGGCCTGGATCTGCG | |
| | CATGCTGCCGTATGGCTGCCTGAGCATTGGCGATTGCGTGGGCCTGAT | |
| | TGAAGTGGTGCGCAACAGCCATACCATTATGCAGATTCAGTGCAAAG | |
| | GCGGCCTGAAAGGCGCGCTGCAGTTTAACAGCCATACCCTGCATCAG | |
| | TGGCTGAAAGATAAAAACAAAGGCGAAATTTATGATGCGGCGATTGA | |
| | TCTGTTTACCCGCAGCTGCGCGGGCTATTGCGTGGCGACCTTTATTCT | |
| | GGGCATTGGCGATCGCCATAACAGCAACATTATGGTGAAAGATGATG | |
| | GCCAGCTGTTTCATATTGATTTTGGCCATTTTCTGGATCATAAAAAAAA | |
| | AAAATTTGGCTATAAACGCGAACGCGTGCCGTTTGTGCTGACCCAGG | |
| | ATTTTCTGATTGTGATTAGCAAAGGCGCGCAGGAATGCACCAAAACC | |
| | CGCGAATTTGAACGCTTTCAGGAAATGTGCTATAAAGCGTATCTGGCG | |
| | ATTCGCCAGCATGCGAACCTGTTTATTAACCTGTTTAGCATGATGCTG | |
| | GGCAGCGGCATGCCGGAACTGCAGAGCTTTGATGATATTGCGTATATT | |
| | CGCAAAACCCTGGCGCTGGATAAAACCGAACAGGAAGCGCTGGAAT | |
| | ATTTTATGAAACAGATGAACGATGCGCATCATGGCGGCTGGACCACC | |
| | AAAATGGATTGGATTTTTCATACCATTAAACAGCATGCGCTGAAC | |
| PIK3CA | MPPRPSSGELWGIHLMPPRILVECLLPNGMIVTLECLREATLITIKHELFK | 3 |
| H1047R | EARKYPLHQLLQDESSYIFVSVTQEAEREEFFDETRRLCDLRLFQPFLKV | |
| Amino Acid | IEPVGNREEKILNREIGFAIGMPVCEFDMVKDPEVQDFRRNILNVCKEAV | |
| Sequence | DLRDLNSPHSRAMYVYPPNVESSPELPKHIYNKLDKGQIIVVIWVIVSPN | |
| | NDKQKYTLKINHDCVPEQVIAEAIRKKTRSMLLSSEQLKLCVLEYQGK | |
| | YILKVCGCDEYFLEKYPLSQYKYIRSCIMLGRMPNLMLMAKESLYSQLP | |
| | MDCFTMPSYSRRISTATPYMNGETSTKSLWVINSALRIKILCATYVNVNI | |
| | RDIDKIYVRTGIYHGGEPLCDNVNTQRVPCSNPRWNEWLNYDIYIPDLP | |
| | RAARLCLSICSVKGRKGAKEEHCPLAWGNINLFDYTDTLVSGKMALNL | |
| | WPVPHGLEDLLNPIGVTGSNPNKETPCLELEFDWFSSVVKFPDMSVIEE | |
| | HANWSVSREAGFSYSHAGLSNRLARDNELRENDKEQLKAISTRDPLSEI | |
| | TEQEKDFLWSHRHYCVTIPEILPKLLLSVKWNSRDEVAQMYCLVKDWP | |
| | PIKPEQAMELLDCNYPDPMVRGFAVRCLEKYLTDDKLSQYLIQLVQVLK | |
| | YEQYLDNLLVRFLLKKALTNQRIGHFFFWHLKSEMHNKTVSQRFGLLL | |
| | ESYCRACGMYLKHLNRQVEAMEKLINLTDILKQEKKDETQKVQMKFL | |
| | VEQMRRPDFMDALQGFLSPLNPAHQLGNLRLEECRIMSSAKRPLWLNW | |
| | ENPDIMSELLFQNNEIIFKNGDDLRQDMLTLQIIRIMENIWQNQGLDLRM | |
| | LPYGCLSIGDCVGLIEVVRNSHTIMQIQCKGGLKGALQFNSHTLHQWLK | |
| | DKNKGEIYDAAIDLFTRSCAGYCVATFILGIGDRHNSNIMVKDDGQLFHI | |
| | DFGHFLDHKKKKFGYKRERVPFVLTQDFLIVISKGAQECTKTREFERFQ | |
| | EMCYKAYLAIRQHANLFINLFSMMLGSGMPELQSFDDIAYIRKTLALDK | |
| | TEQEALEYFMKQMNDARHGGWTTKMDWIFHTIKQHALN | |
| PIK3CA | ATGCCTCCACGACCATCATCAGGTGAACTGTGGGGCATCCACTTGATG | 4 |
| H1047R | CCCCCAAGAATCCTAGTAGAATGTTTACTACCAAATGGAATGATAGTG | |
| Nucleotide | ACTTTAGAATGCCTCCGTGAGGCTACATTAATAACCATAAAGCATGAA | |
| Sequence | CTATTTAAAGAAGCAAGAAAATACCCCCTCCATCAACTTCTTCAAGAT | |
| | GAATCTTCTTACATTTTCGTAAGTGTTACTCAAGAAGCAGAAAGGGA | |
| | AGAATTTTTTGATGAAACAAGACGACTTTGTGACCTTCGGCTTTTTCA | |
| | ACCCTTTTTAAAAGTAATTGAACCAGTAGGCAACCGTGAAGAAAAGA | |
| | TCCTCAATCGAGAAATTGGTTTTGCTATCGGCATGCCAGTGTGTGAAT | |
| | TTGATATGGTTAAAGATCCAGAAGTACAGGACTTCCGAAGAAATATTC | |
| | TGAACGTTTGTAAAGAAGCTGTGGATCTTAGGGACCTCAATTCACCT | |
| | CATAGTAGAGCAATGTATGTCTATCCTCCAAATGTAGAATCTTCACCA | |
| | GAATTGCCAAAGCACATATATAATAAATTAGATAAAGGGCAAATAATA | |
| | GTGGTGATCTGGGTAATAGTTTCTCCAAATAATGACAAGCAGAAGTAT | |
| | ACTCTGAAAATCAACCATGACTGTGTACCAGAACAAGTAATTGCTGA | |
| | AGCAATCAGGAAAAAAACTCGAAGTATGTTGCTATCCTCTGAACAAC | |
| | TAAAACTCTGTGTTTTAGAATATCAGGGCAAGTATATTTTAAAAGTGT | |
| | GTGGATGTGATGAATACTTCCTAGAAAAATATCCTCTGAGTCAGTATA | |
| | AGTATATAAGAAGCTGTATAATGCTTGGGAGGATGCCCAATTTGATGTT | |
| | GATGGCTAAAGAAAGCCTTTATTCTCAACTGCCAATGGACTGTTTTAC | |
| | AATGCCATCTTATTCCAGACGCATTTCCACAGCTACACCATATATGAAT | |
| | GGAGAAACATCTACAAAATCCCTTTGGGTTATAAATAGTGCACTCAGA | |
| | ATAAAAATTCTTTGTGCAACCTACGTGAATGTAAATATTCGAGACATT | |
| | GATAAGATCTATGTTCGAACAGGTATCTACCATGGAGGAGAACCCTTA | |
| | TGTGACAATGTGAACACTCAAAGAGTACCTTGTTCCAATCCCAGGTG | |
| | GAATGAATGGCTGAATTATGATATATACATTCCTGATCTTCCTCGTGCT | |
| | GCTCGACTTTGCCTTTCCATTTGCTCTGTTAAAGGCCGAAAGGGTGCT | |
| | AAAGAGGAACACTGTCCATTGGCATGGGGAAATATAAACTTGTTTGA | |
| | TTACACAGACACTCTAGTATCTGGAAAAATGGCTTTGAATCTTTGGCC | |
| | AGTACCTCATGGATTAGAAGATTTGCTGAACCCTATTGGTGTTACTGG | |
| | ATCAAATCCAAATAAAGAAACTCCATGCTTAGAGTTGGAGTTTGACT | |
| | GGTTCAGCAGTGTGGTAAAGTTCCCAGATATGTCAGTGATTGAAGAG | |
| | CATGCCAATTGGTCTGTATCCCGAGAAGCAGGATTTAGCTATTCCCAC | |
| | GCAGGACTGAGTAACAGACTAGCTAGAGACAATGAATTAAGGGAAA | |
| | ATGACAAAGAACAGCTCAAAGCAATTTCTACACGAGATCCTCTCTCT | |
| | GAAATCACTGAGCAGGAGAAAGATTTTCTATGGAGTCACAGACACTA | |
| | TTGTGTAACTATCCCCGAAATTCTACCCAAATTGCTTCTGTCTGTTAAA | |
| | TGGAATTCTAGAGATGAAGTAGCCCAGATGTATTGCTTGGTAAAAGAT | |
| | TGGCCTCCAATCAAACCTGAACAGGCTATGGAACTTCTGGACTGTAA | |
| | TTACCCAGATCCTATGGTTCGAGGTTTTGCTGTTCGGTGCTTGGAAAA | |
| | ATATTTAACAGATGACAAACTTTCTCAGTATTTAATTCAGCTAGTACAG | |
| | GTCCTAAAATATGAACAATATTTGGATAACTTGCTTGTGAGATTTTTAC | |
| | TGAAGAAAGCATTGACTAATCAAAGGATTGGGCACTTTTTCTTTTGGC | |
| | ATTTAAAATCTGAGATGCACAATAAAACAGTTAGCCAGAGGTTTGGC | |
| | CTGCTTTTGGAGTCCTATTGTCGTGCATGTGGGATGTATTTGAAGCAC | |
| | CTGAATAGGCAAGTCGAGGCAATGGAAAAGCTCATTAACTTAACTGA | |
| | CATTCTCAAACAGGAGAAGAAGGATGAAACACAAAAGGTACAGATG | |
| | AAGTTTTTAGTTGAGCAAATGAGGCGACCAGATTTCATGGATGCTCTA | |
| | CAGGGCTTTCTGTCTCCTCTAAACCCTGCTCATCAACTAGGAAACCTC | |
| | AGGCTTGAAGAGTGTCGAATTATGTCCTCTGCAAAAAGGCCACTGTG | |
| | GTTGAATTGGGAGAACCCAGACATCATGTCAGAGTTACTGTTTCAGA | |
| | ACAATGAGATCATCTTTAAAAATGGGGATGATTTACGGCAAGATATGC | |
| | TAACACTTCAAATTATTCGTATTATGGAAAATATCTGGCAAAATCAAGG | |
| | TCTTGATCTTCGAATGTTACCTTATGGTTGTCTGTCAATCGGTGACTGT | |
| | GTGGGACTTATTGAGGTGGTGCGAAATTCTCACACTATTATGCAAATT | |
| | CAGTGCAAAGGCGGCTTGAAAGGTGCACTGCAGTTCAACAGCCACA | |
| | CACTACATCAGTGGCTCAAAGACAAGAACAAAGGAGAAATATATGAT | |
| | GCAGCCATTGACCTGTTTACACGTTCATGTGCTGGATACTGTGTAGCT | |
| | ACCTTCATTTTGGGAATTGGAGATCGTCACAATAGTAACATCATGGTG | |
| | AAAGACGATGGACAACTGTTTCATATAGATTTTGGACACTTTTTGGAT | |
| | CACAAGAAGAAAAAATTTGGTTATAAACGAGAACGTGTGCCATTTGT | |
| | TTTGACACAGGATTTCTTAATAGTGATTAGTAAAGGAGCCCAAGAATG | |
| | CACAAAGACAAGAGAATTTGAGAGGTTTCAGGAGATGTGTTACAAG | |
| | GCTTATCTAGCTATTCGACAGCATGCCAATCTCTTCATAAATCTTTTCT | |
| | CAATGATGCTTGGCTCTGGAATGCCAGAACTACAATCTTTTGATGACA | |
| | TTGCATACATTCGAAAGACCCTAGCCTTAGATAAAACTGAGCAAGAG | |
| | GCTTTGGAGTATTTCATGAAACAAATGAATGATGCACGTCATGGTGGC | |
| | TGGACAACAAAAATGGATTGGATCTTCCACACAATTAAACAGCATGC | |
| | ATTGAACTGA | |

TABLE 2
KRAS Sequences

| | | SEQ |
|---|---|---|
| | | ID |
| Description | Sequence | NO |
| Wild-type | MTEYKLVVVGAGGVGKSALTIQLIQNHFVDEYDPTIEDSYRKQVVIDGE | 5 |
| KRAS | TCLLDILDTAGQEEYSAMRDQYMRTGEGFLCVFAINNTKSFEDIHHYRE | |
| Amino Acid | QIKRVKDSEDVPMVLVGNKCDLPSRTVDTKQAQDLARSYGIPFIETSAK | |
| Sequence | TRQGVDDAFYTLVREIRKHKEKMSKDGKKKKKKSKTKCVIM | |
| Wild-type | ATGACCGAATATAAACTGGTGGTGGTGGGCGCGGGCGGCGTGGGCAA | 6 |
| KRAS | AAGCGCGCTGACCATTCAGCTGATTCAGAACCATTTTGTGGATGAATA | |
| Nucleotide | TGATCCGACCATTGAAGATAGCTATCGCAAACAGGTGGTGATTGATGG | |
| Sequence | CGAAACCTGCCTGCTGGATATTCTGGATACCGCGGGCCAGGAAGAAT | |
| | ATAGCGCGATGCGCGATCAGTATATGCGCACCGGCGAAGGCTTTCTGT | |
| | GCGTGTTTGCGATTAACAACACCAAAAGCTTTGAAGATATTCATCATT | |
| | ATCGCGAACAGATTAAACGCGTGAAAGATAGCGAAGATGTGCCGATG | |
| | GTGCTGGTGGGCAACAAATGCGATCTGCCGAGCCGCACCGTGGATAC | |
| | CAAACAGGCGCAGGATCTGGCGCGCAGCTATGGCATTCCGTTTATTG | |
| | AAACCAGCGCGAAAACCCGCCAGGGCGTGGATGATGCGTTTTATACC | |
| | CTGGTGCGCGAAATTCGCAAACATAAAGAAAAAATGAGCAAAGATG | |
| | GCAAAAAAAAAAAAAAAAAAAGCAAAACCAAATGCGTGATTATG | |
| KRAS | MTEYKLVVVGAVGVGKSALTIQLIQNHFVDEYDPTIEDSYRKQVVIDGE | 7 |
| G12V | TCLLDILDTAGQEEYSAMRDQYMRTGEGFLCVFAINNTKSFEDIHHYRE | |
| Amino Acid | QIKRVKDSEDVPMVLVGNKCDLPSRTVDTKQAQDLARSYGIPFIETSAK | |
| Sequence | TRQGVDDAFYTLVREIRKHKEKMSKDGKKKKKKSKTKCVIM | |
| KRAS | ATGACCGAATATAAACTGGTGGTGGTGGGCGCGGTGGTGGTGGGCAA | 8 |
| G12V | AAGCGCGCTGACCATTCAGCTGATTCAGAACCATTTTGTGGATGAATA | |
| Nucleotide | TGATCCGACCATTGAAGATAGCTATCGCAAACAGGTGGTGATTGATGG | |
| Sequence | CGAAACCTGCCTGCTGGATATTCTGGATACCGCGGGCCAGGAAGAAT | |
| | ATAGCGCGATGCGCGATCAGTATATGCGCACCGGCGAAGGCTTTCTGT | |
| | GCGTGTTTGCGATTAACAACACCAAAAGCTTTGAAGATATTCATCATT | |
| | ATCGCGAACAGATTAAACGCGTGAAAGATAGCGAAGATGTGCCGATG | |
| | GTGCTGGTGGGCAACAAATGCGATCTGCCGAGCCGCACCGTGGATAC | |
| | CAAACAGGCGCAGGATCTGGCGCGCAGCTATGGCATTCCGTTTATTG | |
| | AAACCAGCGCGAAAACCCGCCAGGGCGTGGATGATGCGTTTTATACC | |
| | CTGGTGCGCGAAATTCGCAAACATAAAGAAAAAATGAGCAAAGATG | |
| | GCAAAAAAAAAAAAAAAAAAAGCAAAACCAAATGCGTGATTATG | |

TABLE 3
p53 Sequences

| | | SEQ |
|---|---|---|
| | | ID |
| Description | Sequence | NO |
| shRNA 1 for | GAATGAGGCCTTGGAATTAAActc | 9 |
| p53 | gagTTTAATTCCAAGGCCTCATTC | |
| shRNA 2 for | TGGCACCTACAGTGAAATTTctcg | 10 |
| p53 | agAAATTTCACTGTAGGTGCCAG | |

TABLE 4
Lentiviral Vector Sequences

| | | SEQ |
|---|---|---|
| | | ID |
| Description | Sequence | NO |
| pHAGE- | TGGAAGGGCTAATTCACTCCCAAAGAAGACAAGATATCCTTGATCTG | 11 |
| PIK3CA- | TGGATCTACCACACACAAGGCTACTTCCCTGATTAGCAGAACTACAC | |
| H1047R | ACCAGGGCCAGGGGTCAGATATCCACTGACCTTTGGATGGTGCTACA | |
| Nucleotide | AGCTAGTACCAGTTGAGCCAGATAAGGTAGAAGAGGCCAATAAAGGA | |
| Sequence | GAGAACACCAGCTTGTTACACCCTGTGAGCCTGCATGGGATGGATGA | |
| | CCCGGAGAGAGAAGTGTTAGAGTGGAGGTTTGACAGCCGCCTAGC | |
| | ATTTCATCACGTGGCCCGAGAGCTGCATCCGGAGTACTTCAAGAACT | |
| | GCTGATATCGAGCTTGCTACAAGGGACTTTCCGCTGGGGACTTTCCA | |
| | GGGAGGCGTGGCCTGGGCGGGACTGGGGAGTGGCGAGCCCTCAGAT | |
| | CCTGCATATAAGCAGCTGCTTTTTGCCTGTACTGGGTCTCTCTGGTTA | |
| | GACCAGATCTGAGCCTGGGAGCTCTCTGGCTAACTAGGGAACCCACT | |
| | GCTTAAGCCTCAATAAAGCTTGCCTTGAGTGCTTCAAGTAGTGTGTGC | |
| | CCGTCTGTTGTGTGACTCTGGTAACTAGAGATCCCTCAGACCCTTTTA | |
| | GTCAGTGTGGAAAATCTCTAGCAGTGGCGCCCGAACAGGGACTTGA | |
| | AAGCGAAAGGGAAACCAGAGGAGCTCTCTCGACGCAGGACTCGGCT | |
| | TGCTGAAGCGCGCACGGCAAGAGGCGAGGGGCGGCGACTGGTGAGT | |
| | ACGCCAAAAATTTTGACTAGCGGAGGCTAGAAGGAGAGAGATGGGT | |
| | GCGAGAGCGTCAGTATTAAGCGGGGGAGAATTAGATCGCGATGGGAA | |
| | AAAATTCGGTTAAGGCCAGGGGGAAAGAAAAAATATAAATTAAAACA | |
| | TATAGTATGGGCAAGCAGGGAGCTAGAACGATTCGCAGTTAATCCTGG | |
| | CCTGTTAGAAACATCAGAAGGCTGTAGACAAATACTGGGACAGCTAC | |
| | AACCATCCCTTCAGACAGGATCAGAAGAACTTAGATCATTATATAATA | |
| | CAGTAGCAACCCTCTATTGTGTGCATCAAAGGATAGAGATAAAAGAC | |
| | ACCAAGGAAGCTTTAGACAAGATAGAGGAAGAGCAAAACAAAAGTA | |
| | AGACCACCGCACAGCAAGCGGCCGGCCGCTGATCTTCAGACCTGGA | |
| | GGAGGAGATATGAGGGACAATTGGAGAAGTGAATTATATAAATATAAA | |
| | GTAGTAAAAATTGAACCATTAGGAGTAGCACCCACCAAGGCAAAGAG | |
| | AAGAGTGGTGCAGAGAGAAAAAAGAGCAGTGGGAATAGGAGCTTTG | |
| | TTCCTTGGGTTCTTGGGAGCAGCAGGAAGCACTATGGGCGCAGCGTC | |
| | AATGACGCTGACGGTACAGGCCAGACAATTATTGTCTGGTATAGTGCA | |
| | GCAGCAGAACAATTTGCTGAGGGCTATTGAGGCGCAACAGCATCTGT | |
| | TGCAACTCACAGTCTGGGGCATCAAGCAGCTCCAGGCAAGAATCCTG | |
| | GCTGTGGAAAGATACCTAAAGGATCAACAGCTCCTGGGGATTTGGGG | |
| | TTGCTCTGGAAAACTCATTTGCACCACTGCTGTGCCTTGGAATGCTAG | |
| | TTGGAGTAATAAATCTCTGGAACAGATTTGGAATCACACGACCTGGAT | |
| | GGAGTGGGACAGAGAAATTAACAATTACACAAGCTTAATACACTCCT | |
| | TAATTGAAGAATCGCAAAACCAGCAAGAAAAGAATGAACAAGAATT | |
| | ATTGGAATTAGATAAATGGGCAAGTTTGTGGAATTGGTTTAACATAAC | |
| | AAATTGGCTGTGGTATATAAAATTATTCATAATGATAGTAGGAGGCTTG | |
| | GTAGGTTTAAGAATAGTTTTTGCTGTACTTTCTATAGTGAATAGAGTTA | |
| | GGCAGGGATATTCACCATTATCGTTTCAGACCCACCTCCCAACCCCGA | |
| | GGGGACCCGACAGGCCCGAAGGAATAGAAGAAGAAGGTGGAGAGA | |
| | GAGACAGAGACAGATCCATTCGATTAGTGAACGGATCTCGACGGTAT | |
| | CGCCGAATTCACAAATGGCAGTATTCATCCACAATTTTAAAAGAAAA | |
| | GGGGGGATTGGGGGGTACAGTGCAGGGGAAAGAATAGTAGACATAAT | |
| | AGCAACAGACATACAAACTAAAGAATTACAAAAACAAATTACAAAA | |
| | ATTCAAAATTTTCGGGTTTATTACAGGGACAGCAGAGATCCAGTTTGG | |
| | ACTAGTCGTGAGGCTCCGGTGCCCGTCAGTGGGCAGAGCGCACATCG | |
| | CCCACAGTCCCCGAGAAGTTGGGGGGAGGGGTCGGCAATTGAACCG | |
| | GTGCCTAGAGAAGGTGGCGCGGGGTAAACTGGGAAAGTGATGTCGT | |
| | GTACTGGCTCCGCCTTTTTCCCGAGGGTGGGGGAGAACCGTATATAA | |
| | GTGCAGTAGTCGCCGTGAACGTTCTTTTTCGCAACGGGTTTGCCGCC | |
| | AGAACACAGGTAAGTGCCGTGTGTGGTTCCCGCGGGCCTGGCCTCTT | |
| | TACGGGTTATGGCCCTTGCGTGCCTTGAATTACTTCCACCTGGCTGCA | |
| | GTACGTGATTCTTGATCCCGAGCTTCGGGTTGGAAGTGGGTGGGAGA | |
| | GTTCGAGGCCTTGCGCTTAAGGAGCCCCTTCGCCTCGTGCTTGAGTT | |
| | GAGGCCTGGCCTGGGCGCTGGGGCCGCCGCGTGCGAATCTGGTGGC | |
| | ACCTTCGCGCCTGTCTCGCTGCTTTCGATAAGTCTCTAGCCATTTAAA | |
| | ATTTTTGATGACCTGCTGCGACGCTTTTTTTCTGGCAAGATAGTCTTGT | |
| | AAATGCGGGCCAAGATCTGCACACTGGTATTTCGGTTTTTGGGGCCG | |
| | CGGGCGGCGACGGGGCCCGTGCGTCCCAGCGCACATGTTCGGCGAG | |
| | GCGGGGCCTGCGAGCGCGGCCACCGAGAATCGGACGGGGGTAGTCT | |
| | CAAGCTGGCCGGCCTGCTCTGGTGCCTGGCCTCGCGCCGCCGTGTAT | |
| | CGCCCCGCCCTGGGCGGCAAGGCTGGCCCGGTCGGCACCAGTTGCG | |
| | TGAGCGGAAAGATGGCCGCTTCCCGGCCCTGCTGCAGGGAGCTCAA | |
| | AATGGAGGACGCGGCGCTCGGGAGAGCGGGGGGTGAGTCACCCAC | |
| | ACAAAGGAAAAGGGCCTTTCCGTCCTCAGCCGTCGCTTCATGTGACT | |
| | CCACGGAGTACCGGGCGCCGTCCAGGCACCTCGATTAGTTCTCGAGC | |
| | TTTTGGAGTACGTCGTCTTTAGGTTGGGGGGAGGGGTTTTATGCGATG | |
| | GAGTTTCCCCACACTGAGTGGGTGGAGACTGAAGTTAGGCCAGCTTG | |
| | GCACTTGATGTAATTCTCCTTGGAATTTGCCCTTTTTGAGTTTGGATCT | |
| | TGGTTCATTCTCAAGCCTCAGACAGTGGTTCAAAGTTTTTTTCTTCCA | |
| | TTTCAGGTGTCGTGAAGCGGCCCTGCAGATATCAACAAGTTTGTACA | |
| | AAAAAGCAGGCACCATGCCTCCAAGACCATCATCAGGTGAACTGTGG | |
| | GGCATCCACTTGATGCCCCCAAGAATCCTAGTAGAATGTTTACTACCA | |
| | AATGGAATGATAGTGACTTTAGAATGCCTCCGTGAGGCTACATTAATA | |
| | ACCATAAAGCATGAACTATTTAAAGAAGCAAGAAAATACCCCCTCCAT | |
| | CAACTTCTTCAAGATGAATCTTCTTACATTTTCGTAAGTGTTACTCAAG | |
| | AAGCAGAAAGGGAAGAATTTTTTGATGAAACAAGACGACTTTGTGA | |
| | CCTTCGGCTTTTTCAACCCTTTTTAAAAGTAATTGAACCAGTAGGCAA | |
| | CCGTGAAGAAAAGATCCTCAATCGAGAAATTGGTTTTGCTATCGGCAT | |
| | GCCAGTGTGTGAATTTGATATGGTTAAAGATCCAGAAGTACAGGACTT | |
| | CCGAAGAAATATTCTGAACGTTTGTAAAGAAGCTGTGGATCTTAGGG | |
| | ACCTCAATTCACCTCATAGTAGAGCAATGTATGTCTATCCTCCAAATGT | |
| | AGAATCTTCACCAGAATTGCCAAAGCACATATATAATAAATTAGATAA | |
| | AGGGCAAATAATAGTGGTGATCTGGGTAATAGTTTCTCCAAATAATGA | |
| | CAAGCAGAAGTATACTCTGAAAATCAACCATGACTGTGTACCAGAAC | |
| | AAGTAATTGCTGAAGCAATCAGGAAAAAAACTCGAAGTATGTTGCTA | |
| | TCCTCTGAACAACTAAAACTCTGTGTTTTAGAATATCAGGGCAAGTAT | |
| | ATTTTAAAAGTGTGTGGATGTGATGAATACTTCCTAGAAAAATATCCT | |
| | CTGAGTCAGTATAAGTATATAAGAAGCTGTATAATGCTTGGGAGGATG | |
| | CCCAATTTGATGTTGATGGCTAAAGAAAGCCTTTATTCTCAACTGCC | |
| | AATGGACTGTTTTACAATGCCATCTTATTCCAGACGCATTTCCACAGC | |
| | TACACCATATATGAATGGAGAAACATCTACAAAATCCCTTTGGGTTATA | |
| | AATAGTGCACTCAGAATAAAAATTCTTTGTGCAACCTACGTGAATGTA | |
| | AATATTCGAGACATTGATAAGATCTATGTTCGAACAGGTATCTACCATG | |
| | GAGGAGAACCCTTATGTGACAATGTGAACACTCAAAGAGTACCTTGT | |
| | TCCAATCCCAGGTGGAATGAATGGCTGAATTATGATATATACATTCCTG | |
| | ATCTTCCTCGTGCTGCTCGACTTTGCCTTTCCATTTGCTCTGTTAAAGG | |
| | CCGAAAGGGTGCTAAAGAGGAACACTGTCCATTGGCATGGGGAAATA | |
| | TAAACTTGTTTGATTACACAGACACTCTAGTATCTGGAAAAATGGCTT | |
| | TGAATCTTTGGCCAGTACCTCATGGATTAGAAGATTTGCTGAACCCTA | |
| | TTGGTGTTACTGGATCAAATCCAAATAAAGAAACTCCATGCTTAGAGT | |
| | TGGAGTTTGACTGGTTCAGCAGTGTGGTAAAGTTCCCAGATATGTCA | |
| | GTGATTGAAGAGCATGCCAATTGGTCTGTATCCCGAGAAGCAGGATTT | |
| | AGCTATTCCCACGCAGGACTGAGTAACAGACTAGCTAGAGACAATGA | |
| | ATTAAGGGAAAATGACAAAGAACAGCTCAAAGCAATTTCTACACGAG | |
| | ATCCTCTCTCTGAAATCACTGAGCAGGAGAAAGATTTTCTATGGAGTC | |
| | ACAGACACTATTGTGTAACTATCCCCGAAATTCTACCCAAATTGCTTC | |
| | TGTCTGTTAAATGGAATTCTAGAGATGAAGTAGCCCAGATGTATTGCT | |
| | TGGTAAAAGATTGGCCTCCAATCAAACCTGAACAGGCTATGGAACTT | |
| | CTGGACTGTAATTACCCAGATCCTATGGTTCGAGGTTTTGCTGTTCGG | |
| | TGCTTGGAAAAATATTTAACAGATGACAAACTTTCTCAGTATTTAATTC | |
| | AGCTAGTACAGGTCCTAAAATATGAACAATATTTGGATAACTTGCTTG | |
| | TGAGATTTTTACTGAAGAAAGCATTGACTAATCAAAGGATTGGGCAC | |
| | TTTTTCTTTTGGCATTTAAAATCTGAGATGCACAATAAAACAGTTAGC | |
| | CAGAGGTTTGGCCTGCTTTTGGAGTCCTATTGTCGTGCATGTGGGATG | |
| | TATTTGAAGCACCTGAATAGGCAAGTCGAGGCAATGGAAAAGCTCAT | |
| | TAACTTAACTGACATTCTCAAACAGGAGAAGAAGGATGAAACACAA | |
| | AAGGTACAGATGAAGTTTTTAGTTGAGCAAATGAGGCGACCAGATTT | |
| | CATGGATGCTCTACAGGGCTTTCTGTCTCCTCTAAACCCTGCTCATCA | |
| | ACTAGGAAACCTCAGGCTTGAAGAGTGTCGAATTATGTCCTCTGCAA | |
| | AAAGGCCACTGTGGTTGAATTGGGAGAACCCAGACATCATGTCAGAG | |
| | TTACTGTTTCAGAACAATGAGATCATCTTTAAAAATGGGGATGATTTA | |
| | CGGCAAGATATGCTAACACTTCAAATTATTCGTATTATGGAAAATATCT | |
| | GGCAAAATCAAGGTCTTGATCTTCGAATGTTACCTTATGGTTGTCTGT | |
| | CAATCGGTGACTGTGTGGGACTTATTGAGGTGGTGCGAAATTCTCAC | |
| | ACTATTATGCAAATTCAGTGCAAAGGCGGCTTGAAAGGTGCACTGCA | |
| | GTTCAACAGCCACACACTACATCAGTGGCTCAAAGACAAGAACAAA | |
| | GGAGAAATATATGATGCAGCCATTGACCTGTTTACACGTTCATGTGCT | |
| | GGATACTGTGTAGCTACCTTCATTTTGGGAATTGGAGATCGTCACAAT | |
| | AGTAACATCATGGTGAAAGACGATGGACAACTGTTTCATATAGATTTT | |
| | GGACACTTTTTGGATCACAAGAAGAAAAAATTTGGTTATAAACGAGA | |
| | ACGTGTGCCATTTGTTTTGACACAGGATTTCTTAATAGTGATTAGTAA | |
| | AGGAGCCCAAGAATGCACAAAGACAAGAGAATTTGAGAGGTTTCAG | |
| | GAGATGTGTTACAAGGCTTATCTAGCTATTCGACAGCATGCCAATCTC | |
| | TTCATAAATCTTTTCTCAATGATGCTTGGCTCTGGAATGCCAGAACTA | |
| | CAATCTTTTGATGACATTGCATACATTCGAAAGACCCTAGCCTTAGATA | |
| | AAACTGAGCAAGAGGCTTTGGAGTATTTCATGAAACAAATGAATGAT | |
| | GCACGTCATGGTGGCTGGACAACAAAAATGGATTGGATCTTCCACAC | |
| | AATTAAACAGCATGCATTGAACTAGAACCCAGCTTTCTTGTACAAAGT | |
| | GGTTGATATCCAGCACAGTGGCGGCCGCTCGAGTCTAGAGGGCCCGC | |
| | GGTTCGAAGGTAAGCCTATCCCTAACCCTCTCCTCGGTCTCGATTCTA | |
| | CGCGTACCGGTTAGTAATGAGATCCCTCCCCCCCCCCTAACGTTACTG | |
| | GCCGAAGCCGCTTGGAATAAGGCCGGTGTGCGTTTGTCTATATGTTAT | |
| | TTTCCACCATATTGCCGTCTTTTGGCAATGTGAGGGCCCGGAAACCTG | |
| | GCCCTGTCTTCTTGACGAGCATTCCTAGGGGTCTTTCCCCTCTCGCCA | |
| | AAGGAATGCAAGGTCTGTTGAATGTCGTGAAGGAAGCAGTTCCTCTG | |
| | GAAGCTTCTTGAAGACAAACAACGTCTGTAGCGACCCTTTGCAGGCA | |
| | GCGGAACCCCCCACCTGGCGACAGGTGCCTCTGCGGCCAAAAGCCA | |
| | CGTGTATAAGATACACCTGCAAAGGCGGCACAACCCCAGTGCCACGT | |
| | TGTGAGTTGGATAGTTGTGGAAAGAGTCAAATGGCTCTCCTCAAGCG | |
| | TATTCAACAAGGGGCTGAAGGATGCCCAGAAGGTACCCCATTGTATG | |
| | GGATCTGATCTGGGGCCTCGGTGCACATGCTTTACATGTGTTTAGTCG | |
| | AGGTTAAAAAAACGTCTAGGCCCCCCGAACCACGGGGACGTGGTTTT | |
| | CCTTTGAAAAACACGATGATAATATGGCCACACATATGACCGAGTACA | |
| | AGCCCACGGTGCGCCTCGCCACCCGCGACGACGTCCCCAGGGCCGT | |
| | ACGCACCCTCGCCGCCGCGTTCGCCGACTACCCCGCCACGCGCCACA | |
| | CCGTCGATCCGGACCGCCACATCGAGCGGGTCACCGAGCTGCAAGA | |
| | ACTCTTCCTCACGCGCGTCGGGCTCGACATCGGCAAGGTGTGGGTCG | |
| | CGGACGACGGCGCCGCGGTGGCGGTCTGGACCACGCCGGAGAGCGT | |
| | CGAAGCGGGGGCGGTGTTCGCCGAGATCGGCCCGCGCATGGCCGAG | |
| | TTGAGCGGTTCCCGGCTGGCCGCGCAGCAACAGATGGAAGGCCTCCT | |
| | GGCGCCGCACCGGCCCAAGGAGCCCGCGTGGTTCCTGGCCACCGTC | |
| | GGCGTCTCGCCCGACCACCAGGGCAAGGGTCTGGGCAGCGCCGTCG | |
| | TGCTCCCCGGAGTGGAGGCGGCCGAGCGCGCCGGGGTGCCCGCCTT | |
| | CCTGGAGACCTCCGCGCCCCGCAACCTCCCCTTCTACGAGCGGCTCG | |
| | GCTTCACCGTCACCGCCGACGTCGAGGTGCCCGAAGGACCGCGCAC | |
| | CTGGTGCATGACCCGCAAGCCCGGTGCCTAAATCGATAGATCCTAATC | |
| | AACCTCTGGATTACAAAATTTGTGAAAGATTGACTGGTATTCTTAACT | |
| | ATGTTGCTCCTTTTACGCTATGTGGATACGCTGCTTTAATGCCTTTGTAT | |
| | CATGCTATTGCTTCCCGTATGGCTTTCATTTTCTCCTCCTTGTATAAATC | |
| | CTGGTTGCTGTCTCTTTATGAGGAGTTGTGGCCCGTTGTCAGGCAACG | |
| | TGGCGTGGTGTGCACTGTGTTTGCTGACGCAACCCCCACTGGTTGGG | |
| | GCATTGCCACCACCTGTCAGCTCCTTTCCGGGACTTTCGCTTTCCCCC | |
| | TCCCTATTGCCACGGCGGAACTCATCGCCGCCTGCCTTGCCCGCTGC | |
| | TGGACAGGGGCTCGGCTGTTGGGCACTGACAATTCCGTGGTGTTGTC | |
| | GGGGAAATCATCGTCCTTTCCTTGGCTGCTCGCCTGTGTTGCCACCTG | |
| | GATTCTGCGCGGGACGTCCTTCTGCTACGTCCCTTCGGCCCTCAATCC | |
| | AGCGGACCTTCCTTCCCGCGGCCTGCTGCCGGCTCTGCGGCCTCTTC | |
| | CGCGTCTTCGCCTTCGCCCTCAGACGAGTCGGATCTCCCTTTGGGCC | |
| | GCCTCCCCGCCTGAGATCCTTTAAGACCAATGACTTACAAGGCAGCT | |
| | GTAGATCTTAGCCACTTTTTAAAAGAAAAGGGGGGACTGGAAGGGCT | |
| | AATTCACTCCCAACGAAGACAAGATCTGCTTTTTGCTTGTACTGGGTC | |
| | TCTCTGGTTAGACCAGATCTGAGCCTGGGAGCTCTCTGGCTAACTAG | |
| | GGAACCCACTGCTTAAGCCTCAATAAAGCTTGCCTTGAGTGCTTCAA | |
| | GTAGTGTGTGCCCGTCTGTTGTGTGACTCTGGTAACTAGAGATCCCTC | |
| | AGACCCTTTTAGTCAGTGTGGAAAATCTCTAGCAGTAGTAGTTCATGT | |
| | CATCTTATTATTCAGTATTTATAACTTGCAAAGAAATGAATATCAGAGA | |
| | GTGAGAGGCCCGGGTTAATTAAGGAAAGGGCTAGATCATTCTTGAAG | |
| | ACGAAAGGGCCTCGTGATACGCCTATTTTTATAGGTTAATGTCATGATA | |
| | ATAATGGTTTCTTAGACGTCAGGTGGCACTTTTCGGGGAAATGTGCGC | |
| | GGAACCCCTATTTGTTTATTTTTCTAAATACATTCAAATATGTATCCGCT | |
| | CATGAGACAATAACCCTGATAAATGCTTCAATAATATTGAAAAAGGAA | |
| | GAGTATGAGTATTCAACATTTCCGTGTCGCCCTTATTCCCTTTTTTGCG | |
| | GCATTTTGCCTTCCTGTTTTTGCTCACCCAGAAACGCTGGTGAAAGTA | |
| | AAAGATGCTGAAGATCAGTTGGGTGCACGAGTGGGTTACATCGAACT | |
| | GGATCTCAACAGCGGTAAGATCCTTGAGAGTTTTCGCCCCGAAGAA | |
| | CGTTTTCCAATGATGAGCACTTTTAAAGTTCTGCTATGTGGCGCGGTA | |
| | TTATCCCGTGTTGACGCCGGGCAAGAGCAACTCGGTCGCCGCATACA | |
| | CTATTCTCAGAATGACTTGGTTGAGTACTCACCAGTCACAGAAAAGC | |
| | ATCTTACGGATGGCATGACAGTAAGAGAATTATGCAGTGCTGCCATAA | |
| | CCATGAGTGATAACACTGCGGCCAACTTACTTCTGACAACGATCGGA | |
| | GGACCGAAGGAGCTAACCGCTTTTTTGCACAACATGGGGGATCATGT | |
| | AACTCGCCTTGATCGTTGGGAACCGGAGCTGAATGAAGCCATACCAA | |
| | ACGACGAGCGTGACACCACGATGCCTGTAGCAATGGCAACAACGTTG | |
| | CGCAAACTATTAACTGGCGAACTACTTACTCTAGCTTCCCGGCAACAA | |
| | TTAATAGACTGGATGGAGGCGGATAAAGTTGCAGGACCACTTCTGCG | |
| | CTCGGCCCTTCCGGCTGGCTGGTTTATTGCTGATAAATCTGGAGCCGG | |
| | TGAGCGTGGGTCTCGCGGTATCATTGCAGCACTGGGGCCAGATGGTA | |
| | AGCCCTCCCGTATCGTAGTTATCTACACGACGGGGAGTCAGGCAACT | |
| | ATGGATGAACGAAATAGACAGATCGCTGAGATAGGTGCCTCACTGAT | |
| | TAAGCATTGGTAACTGTCAGACCAAGTTTACTCATATATACTTTAGATT | |
| | GATTTAAAACTTCATTTTTAATTTAAAAGGATCTAGGTGAAGATCCTTT | |
| | TTGATAATCTCATGACCAAAATCCCTTAACGTGAGTTTTCGTTCCACT | |
| | GAGCGTCAGACCCCGTAGAAAAGATCAAAGGATCTTCTTGAGATCCT | |
| | TTTTTTCTGCGCGTAATCTGCTGCTTGCAAACAAAAAAACCACCGCTA | |
| | CCAGCGGTGGTTTGTTTGCCGGATCAAGAGCTACCAACTCTTTTTCCG | |
| | AAGGTAACTGGCTTCAGCAGAGCGCAGATACCAAATACTGTTCTTCT | |
| | AGTGTAGCCGTAGTTAGGCCACCACTTCAAGAACTCTGTAGCACCGC | |
| | CTACATACCTCGCTCTGCTAATCCTGTTACCAGTGGCTGCTGCCAGTG | |
| | GCGATAAGTCGTGTCTTACCGGGTTGGACTCAAGACGATAGTTACCG | |
| | GATAAGGCGCAGCGGTCGGGCTGAACGGGGGGTTCGTGCACACAGC | |
| | CCAGCTTGGAGCGAACGACCTACACCGAACTGAGATACCTACAGCGT | |
| | GAGCTATGAGAAAGCGCCACGCTTCCCGAAGGGAGAAAGGCGGACA | |
| | GGTATCCGGTAAGCGGCAGGGTCGGAACAGGAGAGCGCACGAGGGA | |
| | GCTTCCAGGGGGAAACGCCTGGTATCTTTATAGTCCTGTCGGGTTTCG | |
| | CCACCTCTGACTTGAGCGTCGATTTTTGTGATGCTCGTCAGGGGGGC | |
| | GGAGCCTATGGAAAAACGCCAGCAACGCGGCCTTTTTACGGTTCCTG | |
| | GCCTTTTGCTGGCCTTTTGCTCACATGTTCTTTCCTGCGTTATCCCCTG | |
| | ATTCTGTGGATAACCGTATTACCGCCTTTGAGTGAGCTGATACCGCTC | |
| | GCCGCAGCCGAACGACCGAGCGCAGCGAGTCAGTGAGCGAGGAAG | |
| | CGGAAGAGCGCCCAATACGCAAACCGCCTCTCCCCGCGCGTTGGCCG | |
| | ATTCATTAATGCAGCAAGCTCATGGCTGACTAATTTTTTTTATTTATGC | |
| | AGAGGCCGAGGCCGCCTCGGCCTCTGAGCTATTCCAGAAGTAGTGAG | |
| | GAGGCTTTTTTGGAGGCCTAGGCTTTTGCAAAAAGCTCCCCGTGGCA | |
| | CGACAGGTTTCCCGACTGGAAAGCGGGCAGTGAGCGCAACGCAATT | |
| | AATGTGAGTTAGCTCACTCATTAGGCACCCCAGGCTTTACACTTTATG | |
| | CTTCCGGCTCGTATGTTGTGTGGAATTGTGAGCGGATAACAATTTCAC | |
| | ACAGGAAACAGCTATGACATGATTACGAATTTCACAAATAAAGCATTT | |
| | TTTTCACTGCATTCTAGTTGTGGTTTGTCCAAACTCATCAATGTATCTT | |
| | ATCATGTCTGGATCAACTGGATAACTCAAGCTAACCAAAATCATCCCA | |
| | AACTTCCCACCCCATACCCTATTACCACTGCCAATTACCTGTGGTTTCA | |
| | TTTACTCTAAACCTGTGATTCCTCTGAATTATTTTCATTTTAAAGAAAT | |
| | TGTATTTGTTAAATATGTACTACAAACTTAGTAGT | |
| pHAGE- | TGGAAGGGCTAATTCACTCCCAAAGAAGACAAGATATCCTTGATCTG | 12 |
| KRAS- | TGGATCTACCACACACAAGGCTACTTCCCTGATTAGCAGAACTACAC | |
| G12V | ACCAGGGCCAGGGGTCAGATATCCACTGACCTTTGGATGGTGCTAC | |
| Nucleotide | AAGCTAGTACCAGTTGAGCCAGATAAGGTAGAAGAGGCCAATAAAGG | |
| Sequence | AGAGAACACCAGCTTGTTACACCCTGTGAGCCTGCATGGGATGGATG | |
| | ACCCGGAGAGAGAAGTGTTAGAGTGGAGGTTTGACAGCCGCCTAGC | |
| | ATTTCATCACGTGGCCCGAGAGCTGCATCCGGAGTACTTCAAGAACT | |
| | GCTGATATCGAGCTTGCTACAAGGGACTTTCCGCTGGGGACTTTCCA | |
| | GGGAGGCGTGGCCTGGGCGGGACTGGGGAGTGGCGAGCCCTCAGAT | |
| | CCTGCATATAAGCAGCTGCTTTTTGCCTGTACTGGGTCTCTCTGGTTA | |
| | GACCAGATCTGAGCCTGGGAGCTCTCTGGCTAACTAGGGAACCCACT | |
| | GCTTAAGCCTCAATAAAGCTTGCCTTGAGTGCTTCAAGTAGTGTGTGC | |
| | CCGTCTGTTGTGTGACTCTGGTAACTAGAGATCCCTCAGACCCTTTTA | |
| | GTCAGTGTGGAAAATCTCTAGCAGTGGCGCCCGAACAGGGACTTGA | |
| | AAGCGAAAGGGAAACCAGAGGAGCTCTCTCGACGCAGGACTCGGCT | |
| | TGCTGAAGCGCGCACGGCAAGAGGCGAGGGGCGGCGACTGGTGAGT | |
| | ACGCCAAAAATTTTGACTAGCGGAGGCTAGAAGGAGAGAGATGGGT | |
| | GCGAGAGCGTCAGTATTAAGCGGGGGAGAATTAGATCGCGATGGGAA | |
| | AAAATTCGGTTAAGGCCAGGGGGAAAGAAAAAATATAAATTAAAACA | |
| | TATAGTATGGGCAAGCAGGGAGCTAGAACGATTCGCAGTTAATCCTGG | |
| | CCTGTTAGAAACATCAGAAGGCTGTAGACAAATACTGGGACAGCTAC | |
| | AACCATCCCTTCAGACAGGATCAGAAGAACTTAGATCATTATATAATA | |
| | CAGTAGCAACCCTCTATTGTGTGCATCAAAGGATAGAGATAAAAGAC | |
| | ACCAAGGAAGCTTTAGACAAGATAGAGGAAGAGCAAAACAAAAGTA | |
| | AGACCACCGCACAGCAAGCGGCCGGCCGCTGATCTTCAGACCTGGA | |
| | GGAGGAGATATGAGGGACAATTGGAGAAGTGAATTATATAAATATAAA | |
| | GTAGTAAAAATTGAACCATTAGGAGTAGCACCCACCAAGGCAAAGAG | |
| | AAGAGTGGTGCAGAGAGAAAAAAGAGCAGTGGGAATAGGAGCTTTG | |
| | TTCCTTGGGTTCTTGGGAGCAGCAGGAAGCACTATGGGCGCAGCGTC | |
| | AATGACGCTGACGGTACAGGCCAGACAATTATTGTCTGGTATAGTGCA | |
| | GCAGCAGAACAATTTGCTGAGGGCTATTGAGGCGCAACAGCATCTGT | |
| | TGCAACTCACAGTCTGGGGCATCAAGCAGCTCCAGGCAAGAATCCTG | |
| | GCTGTGGAAAGATACCTAAAGGATCAACAGCTCCTGGGGATTTGGGG | |
| | TTGCTCTGGAAAACTCATTTGCACCACTGCTGTGCCTTGGAATGCTAG | |
| | TTGGAGTAATAAATCTCTGGAACAGATTTGGAATCACACGACCTGGAT | |
| | GGAGTGGGACAGAGAAATTAACAATTACACAAGCTTAATACACTCCT | |
| | TAATTGAAGAATCGCAAAACCAGCAAGAAAAGAATGAACAAGAATT | |
| | ATTGGAATTAGATAAATGGGCAAGTTTGTGGAATTGGTTTAACATAAC | |
| | AAATTGGCTGTGGTATATAAAATTATTCATAATGATAGTAGGAGGCTTG | |
| | GTAGGTTTAAGAATAGTTTTTGCTGTACTTTCTATAGTGAATAGAGTTA | |
| | GGCAGGGATATTCACCATTATCGTTTCAGACCCACCTCCCAACCCCGA | |
| | GGGGACCCGACAGGCCCGAAGGAATAGAAGAAGAAGGTGGAGAGA | |
| | GAGACAGAGACAGATCCATTCGATTAGTGAACGGATCTCGACGGTAT | |
| | CGCCGAATTCACAAATGGCAGTATTCATCCACAATTTTAAAAGAAAA | |
| | GGGGGGATTGGGGGGTACAGTGCAGGGGAAAGAATAGTAGACATAAT | |
| | AGCAACAGACATACAAACTAAAGAATTACAAAAACAAATTACAAAA | |
| | ATTCAAAATTTTCGGGTTTATTACAGGGACAGCAGAGATCCAGTTTGG | |
| | ACTAGTCGTGAGGCTCCGGTGCCCGTCAGTGGGCAGAGCGCACATCG | |
| | CCCACAGTCCCCGAGAAGTTGGGGGGAGGGGTCGGCAATTGAACCG | |
| | GTGCCTAGAGAAGGTGGCGCGGGGTAAACTGGGAAAGTGATGTCGT | |
| | GTACTGGCTCCGCCTTTTTCCCGAGGGTGGGGGAGAACCGTATATAA | |
| | GTGCAGTAGTCGCCGTGAACGTTCTTTTTCGCAACGGGTTTGCCGCC | |
| | AGAACACAGGTAAGTGCCGTGTGTGGTTCCCGCGGGCCTGGCCTCTT | |
| | TACGGGTTATGGCCCTTGCGTGCCTTGAATTACTTCCACCTGGCTGCA | |
| | GTACGTGATTCTTGATCCCGAGCTTCGGGTTGGAAGTGGGTGGGAGA | |
| | GTTCGAGGCCTTGCGCTTAAGGAGCCCCTTCGCCTCGTGCTTGAGTT | |
| | GAGGCCTGGCCTGGGCGCTGGGGCCGCCGCGTGCGAATCTGGTGGC | |
| | ACCTTCGCGCCTGTCTCGCTGCTTTCGATAAGTCTCTAGCCATTTAAA | |
| | ATTTTTGATGACCTGCTGCGACGCTTTTTTTCTGGCAAGATAGTCTTGT | |
| | AAATGCGGGCCAAGATCTGCACACTGGTATTTCGGTTTTTGGGGCCG | |
| | CGGGCGGCGACGGGGCCCGTGCGTCCCAGCGCACATGTTCGGCGAG | |
| | GCGGGGCCTGCGAGCGCGGCCACCGAGAATCGGACGGGGGTAGTCT | |
| | CAAGCTGGCCGGCCTGCTCTGGTGCCTGGCCTCGCGCCGCCGTGTAT | |
| | CGCCCCGCCCTGGGCGGCAAGGCTGGCCCGGTCGGCACCAGTTGCG | |
| | TGAGCGGAAAGATGGCCGCTTCCCGGCCCTGCTGCAGGGAGCTCAA | |
| | AATGGAGGACGCGGCGCTCGGGAGAGCGGGCGGGTGAGTCACCCAC | |
| | ACAAAGGAAAAGGGCCTTTCCGTCCTCAGCCGTCGCTTCATGTGACT | |
| | CCACGGAGTACCGGGCGCCGTCCAGGCACCTCGATTAGTTCTCGAGC | |
| | TTTTGGAGTACGTCGTCTTTAGGTTGGGGGGAGGGGTTTTATGCGATG | |
| | GAGTTTCCCCACACTGAGTGGGTGGAGACTGAAGTTAGGCCAGCTTG | |
| | GCACTTGATGTAATTCTCCTTGGAATTTGCCCTTTTTGAGTTTGGATCT | |
| | TGGTTCATTCTCAAGCCTCAGACAGTGGTTCAAAGTTTTTTTCTTCCA | |
| | TTTCAGGTGTCGTGAAGCGGCCCTGCAGATATCAACAAGTTTGTACA | |
| | AAAAAGCAGGCACCATGACTGAATATAAACTTGTGGTAGTTGGAGCT | |
| | GTTGGCGTAGGCAAGAGTGCCTTGACGATACAGCTAATTCAGAATCAT | |
| | TTTGTGGACGAATATGATCCAACAATAGAGGATTCCTACAGGAAGCA | |
| | AGTAGTAATTGATGGAGAAACCTGTCTCTTGGATATTCTCGACACAGC | |
| | AGGTCAAGAGGAGTACAGTGCAATGAGGGACCAGTACATGAGGACT | |
| | GGGGAGGGCTTTCTTTGTGTATTTGCCATAAATAATACTAAATCATTTG | |
| | AAGATATTCACCATTATAGAGAACAAATTAAAAGAGTTAAGGACTCTG | |
| | AAGATGTACCTATGGTCCTAGTAGGAAATAAATGTGATTTGCCTTCTA | |
| | GAACAGTAGACACAAAACAGGCTCAGGACTTAGCAAGAAGTTATGG | |
| | AATTCCTTTTATTGAAACATCAGCAAAGACAAGACAGGGTGTTGATG | |
| | ATGCCTTCTATACATTAGTTCGAGAAATTCGAAAACATAAAGAAAAGA | |
| | TGAGCAAAGATGGTAAAAAGAAGAAAAAGAAGTCAAAGACAAAGT | |
| | GTGTAATTATGTAGAACCCAGCTTTCTTGTACAAAGTGGTTGATATCC | |
| | AGCACAGTGGCGGCCGCTCGAGTCTAGAGGGCCCGCGGTTCGAAGG | |
| | TAAGCCTATCCCTAACCCTCTCCTCGGTCTCGATTCTACGCGTACCGG | |
| | TTAGTAATGAGATCCCTCCCCCCCCCCTAACGTTACTGGCCGAAGCCG | |
| | CTTGGAATAAGGCCGGTGTGCGTTTGTCTATATGTTATTTTCCACCATA | |
| | TTGCCGTCTTTTGGCAATGTGAGGGCCCGGAAACCTGGCCCTGTCTT | |
| | CTTGACGAGCATTCCTAGGGGTCTTTCCCCTCTCGCCAAAGGAATGC | |
| | AAGGTCTGTTGAATGTCGTGAAGGAAGCAGTTCCTCTGGAAGCTTCT | |
| | TGAAGACAAACAACGTCTGTAGCGACCCTTTGCAGGCAGCGGAACC | |
| | CCCCACCTGGCGACAGGTGCCTCTGCGGCCAAAAGCCACGTGTATAA | |
| | GATACACCTGCAAAGGCGGCACAACCCCAGTGCCACGTTGTGAGTTG | |
| | GATAGTTGTGGAAAGAGTCAAATGGCTCTCCTCAAGCGTATTCAACA | |
| | AGGGGCTGAAGGATGCCCAGAAGGTACCCCATTGTATGGGATCTGAT | |
| | CTGGGGCCTCGGTGCACATGCTTTACATGTGTTTAGTCGAGGTTAAAA | |
| | AAACGTCTAGGCCCCCCGAACCACGGGGACGTGGTTTTCCTTTGAAA | |
| | AACACGATGATAATATGGCCACACATATGGTGAGCAAGGGCGAGGAG | |
| | CTGTTCACCGGGGTGGTGCCCATCCTGGTCGAGCTGGACGGCGACGT | |
| | AAACGGCCACAAGTTCAGCGTGTCCGGCGAGGGCGAGGGCGATGCC | |
| | ACCTACGGCAAGCTGACCCTGAAGTTCATCTGCACCACCGGCAAGCT | |
| | GCCCGTGCCCTGGCCCACCCTCGTGACCACCCTGACCTACGGCGTGC | |
| | AGTGCTTCAGCCGCTACCCCGACCACATGAAGCAGCACGACTTCTTC | |
| | AAGTCCGCCATGCCCGAAGGCTACGTCCAGGAGCGCACCATCTTCTT | |
| | CAAGGACGACGGCAACTACAAGACCCGCGCCGAGGTGAAGTTCGAG | |
| | GGCGACACCCTGGTGAACCGCATCGAGCTGAAGGGCATCGACTTCA | |
| | AGGAGGACGGCAACATCCTGGGGCACAAGCTGGAGTACAACTACAA | |
| | CAGCCACAACGTCTATATCATGGCCGACAAGCAGAAGAACGGCATCA | |
| | AGGTGAACTTCAAGATCCGCCACAACATCGAGGACGGCAGCGTGCA | |
| | GCTCGCCGACCACTACCAGCAGAACACCCCCATCGGCGACGGCCCCG | |
| | TGCTGCTGCCCGACAACCACTACCTGAGCACCCAGTCCGCCCTGAGC | |
| | AAAGACCCCAACGAGAAGCGCGATCACATGGTCCTGCTGGAGTTCGT | |
| | GACCGCCGCCGGGATCACTCTCGGCATGGACGAGCTGTACAAGTAAA | |
| | TCGATAGATCCTAATCAACCTCTGGATTACAAAATTTGTGAAAGATTG | |
| | ACTGGTATTCTTAACTATGTTGCTCCTTTTACGCTATGTGGATACGCTG | |
| | CTTTAATGCCTTTGTATCATGCTATTGCTTCCCGTATGGCTTTCATTTTC | |
| | TCCTCCTTGTATAAATCCTGGTTGCTGTCTCTTTATGAGGAGTTGTGGC | |
| | CCGTTGTCAGGCAACGTGGCGTGGTGTGCACTGTGTTTGCTGACGCA | |
| | ACCCCCACTGGTTGGGGCATTGCCACCACCTGTCAGCTCCTTTCCGG | |
| | GACTTTCGCTTTCCCCCTCCCTATTGCCACGGCGGAACTCATCGCCGC | |
| | CTGCCTTGCCCGCTGCTGGACAGGGGCTCGGCTGTTGGGCACTGACA | |
| | ATTCCGTGGTGTTGTCGGGGAAATCATCGTCCTTTCCTTGGCTGCTCG | |
| | CCTGTGTTGCCACCTGGATTCTGCGCGGGACGTCCTTCTGCTACGTCC | |
| | CTTCGGCCCTCAATCCAGCGGACCTTCCTTCCCGCGGCCTGCTGCCG | |
| | GCTCTGCGGCCTCTTCCGCGTCTTCGCCTTCGCCCTCAGACGAGTCG | |
| | GATCTCCCTTTGGGCCGCCTCCCCGCCTGAGATCCTTTAAGACCAATG | |
| | ACTTACAAGGCAGCTGTAGATCTTAGCCACTTTTTAAAAGAAAAGGG | |
| | GGGACTGGAAGGGCTAATTCACTCCCAACGAAGACAAGATCTGCTTT | |
| | TTGCTTGTACTGGGTCTCTCTGGTTAGACCAGATCTGAGCCTGGGAGC | |
| | TCTCTGGCTAACTAGGGAACCCACTGCTTAAGCCTCAATAAAGCTTGC | |
| | CTTGAGTGCTTCAAGTAGTGTGTGCCCGTCTGTTGTGTGACTCTGGTA | |
| | ACTAGAGATCCCTCAGACCCTTTTAGTCAGTGTGGAAAATCTCTAGCA | |
| | GTAGTAGTTCATGTCATCTTATTATTCAGTATTTATAACTTGCAAAGAA | |
| | ATGAATATCAGAGAGTGAGAGGCCCGGGTTAATTAAGGAAAGGGCTA | |
| | GATCATTCTTGAAGACGAAAGGGCCTCGTGATACGCCTATTTTTATAG | |
| | GTTAATGTCATGATAATAATGGTTTCTTAGACGTCAGGTGGCACTTTTC | |
| | GGGGAAATGTGCGCGGAACCCCTATTTGTTTATTTTTCTAAATACATTC | |
| | AAATATGTATCCGCTCATGAGACAATAACCCTGATAAATGCTTCAATAA | |
| | TATTGAAAAAGGAAGAGTATGAGTATTCAACATTTCCGTGTCGCCCTT | |
| | ATTCCCTTTTTTGCGGCATTTTGCCTTCCTGTTTTTGCTCACCCAGAAA | |
| | CGCTGGTGAAAGTAAAAGATGCTGAAGATCAGTTGGGTGCACGAGT | |
| | GGGTTACATCGAACTGGATCTCAACAGCGGTAAGATCCTTGAGAGTT | |
| | TTCGCCCCGAAGAACGTTTTCCAATGATGAGCACTTTTAAAGTTCTGC | |
| | TATGTGGCGCGGTATTATCCCGTGTTGACGCCGGGCAAGAGCAACTC | |
| | GGTCGCCGCATACACTATTCTCAGAATGACTTGGTTGAGTACTCACCA | |
| | GTCACAGAAAAGCATCTTACGGATGGCATGACAGTAAGAGAATTATG | |
| | CAGTGCTGCCATAACCATGAGTGATAACACTGCGGCCAACTTACTTCT | |
| | GACAACGATCGGAGGACCGAAGGAGCTAACCGCTTTTTTGCACAAC | |
| | ATGGGGGATCATGTAACTCGCCTTGATCGTTGGGAACCGGAGCTGAA | |
| | TGAAGCCATACCAAACGACGAGCGTGACACCACGATGCCTGTAGCAA | |
| | TGGCAACAACGTTGCGCAAACTATTAACTGGCGAACTACTTACTCTA | |
| | GCTTCCCGGCAACAATTAATAGACTGGATGGAGGCGGATAAAGTTGC | |
| | AGGACCACTTCTGCGCTCGGCCCTTCCGGCTGGCTGGTTTATTGCTGA | |
| | TAAATCTGGAGCCGGTGAGCGTGGGTCTCGCGGTATCATTGCAGCAC | |
| | TGGGGCCAGATGGTAAGCCCTCCCGTATCGTAGTTATCTACACGACGG | |
| | GGAGTCAGGCAACTATGGATGAACGAAATAGACAGATCGCTGAGATA | |
| | GGTGCCTCACTGATTAAGCATTGGTAACTGTCAGACCAAGTTTACTCA | |
| | TATATACTTTAGATTGATTTAAAACTTCATTTTTAATTTAAAAGGATCTA | |
| | GGTGAAGATCCTTTTTGATAATCTCATGACCAAAATCCCTTAACGTGA | |
| | GTTTTCGTTCCACTGAGCGTCAGACCCCGTAGAAAAGATCAAAGGA | |
| | TCTTCTTGAGATCCTTTTTTTCTGCGCGTAATCTGCTGCTTGCAAACA | |
| | AAAAAACCACCGCTACCAGCGGTGGTTTGTTTGCCGGATCAAGAGCT | |
| | ACCAACTCTTTTTCCGAAGGTAACTGGCTTCAGCAGAGCGCAGATAC | |
| | CAAATACTGTTCTTCTAGTGTAGCCGTAGTTAGGCCACCACTTCAAGA | |
| | ACTCTGTAGCACCGCCTACATACCTCGCTCTGCTAATCCTGTTACCAG | |
| | TGGCTGCTGCCAGTGGCGATAAGTCGTGTCTTACCGGGTTGGACTCA | |
| | AGACGATAGTTACCGGATAAGGCGCAGCGGTCGGGCTGAACGGGGG | |
| | GTTCGTGCACACAGCCCAGCTTGGAGCGAACGACCTACACCGAACT | |
| | GAGATACCTACAGCGTGAGCTATGAGAAAGCGCCACGCTTCCCGAAG | |
| | GGAGAAAGGCGGACAGGTATCCGGTAAGCGGCAGGGTCGGAACAGG | |
| | AGAGCGCACGAGGGAGCTTCCAGGGGGAAACGCCTGGTATCTTTATA | |
| | GTCCTGTCGGGTTTCGCCACCTCTGACTTGAGCGTCGATTTTTGTGAT | |
| | GCTCGTCAGGGGGGCGGAGCCTATGGAAAAACGCCAGCAACGCGGC | |
| | CTTTTTACGGTTCCTGGCCTTTTGCTGGCCTTTTGCTCACATGTTCTTT | |
| | CCTGCGTTATCCCCTGATTCTGTGGATAACCGTATTACCGCCTTTGAGT | |
| | GAGCTGATACCGCTCGCCGCAGCCGAACGACCGAGCGCAGCGAGTC | |
| | AGTGAGCGAGGAAGCGGAAGAGCGCCCAATACGCAAACCGCCTCTC | |
| | CCCGCGCGTTGGCCGATTCATTAATGCAGCAAGCTCATGGCTGACTAA | |
| | TTTTTTTTATTTATGCAGAGGCCGAGGCCGCCTCGGCCTCTGAGCTATT | |
| | CCAGAAGTAGTGAGGAGGCTTTTTTGGAGGCCTAGGCTTTTGCAAAA | |
| | AGCTCCCCGTGGCACGACAGGTTTCCCGACTGGAAAGCGGGCAGTG | |
| | AGCGCAACGCAATTAATGTGAGTTAGCTCACTCATTAGGCACCCCAG | |
| | GCTTTACACTTTATGCTTCCGGCTCGTATGTTGTGTGGAATTGTGAGC | |
| | GGATAACAATTTCACACAGGAAACAGCTATGACATGATTACGAATTTC | |
| | ACAAATAAAGCATTTTTTTCACTGCATTCTAGTTGTGGTTTGTCCAAA | |
| | CTCATCAATGTATCTTATCATGTCTGGATCAACTGGATAACTCAAGCTA | |
| | ACCAAAATCATCCCAAACTTCCCACCCCATACCCTATTACCACTGCCA | |
| | ATTACCTGTGGTTTCATTTACTCTAAACCTGTGATTCCTCTGAATTATTT | |
| | TCATTTTAAAGAAATTGTATTTGTTAAATATGTACTACAAACTTAGTAG | |
| | T | |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

Those skilled in the art will recognize, or be able to ascertain using no more than routine experimentation, numerous equivalents to the compounds and methods of use thereof described herein. Such equivalents are considered to be within the scope of this invention and are covered by the following claims. Those skilled in the art will also recognize that all combinations of embodiments described herein are within the scope of the invention.

1. An orthotopic non-human mammal model of ovarian cancer comprising ovarian tissue with at least one cancer cell, wherein the ovarian tissue expresses an exogenous mutant PIK3CA polypeptide and an exogenous mutant KRAS polypeptide, and wherein the ovarian tissue has reduced expression of endogenous p53.

2. An orthotopic non-human mammal model of ovarian cancer comprising ovarian tissue with at least one cancer cell induced by expression of an exogenous mutant PIK3CA polypeptide and an exogenous mutant KRAS polypeptide, and reduced expression of endogenous p53.

3. The orthotopic non-human mammal model of claim 1, wherein the mammal is porcine.

4. The orthotopic non-human mammal model of claim 1, wherein the mutant PIK3CA polypeptide comprises an H1047R amino acid substitution.

5. The orthotopic non-human mammal model of claim 4, wherein the mutant PIK3CA polypeptide comprises an amino acid sequence at least 90% identical to SEQ ID NO: 1.

6. The orthotopic non-human mammal model of claim 1, wherein the mutant KRAS polypeptide comprises a G12V amino acid substitution.

7. The orthotopic non-human mammal model of claim 6, wherein the mutant KRAS polypeptide comprises an amino acid sequence at least 90% identical to SEQ ID NO: 5.

8. The orthotopic non-human mammal model of claim 1, wherein the ovarian tissue comprises a nucleotide sequence encoding the exogenous mutant PIK3CA polypeptide.

9. The orthotopic non-human mammal model of claim 8, wherein the nucleotide sequence encoding the exogenous mutant PIK3CA polypeptide comprises a sequence at least 90% identical to SEQ ID NO: 2.

10. The orthotopic non-human mammal model of claim 1, wherein the ovarian tissue comprises a nucleotide sequence encoding the exogenous mutant KRAS polypeptide.

11. The orthotopic non-human mammal model of claim 10, wherein the nucleotide sequence encoding the exogenous mutant KRAS polypeptide comprises a sequence at least 90% identical to SEQ ID NO: 6.

12. The orthotopic non-human mammal model of claim 1, wherein expression of p53 is reduced by compared to ovarian tissue of a wild-type mammal.

13. The orthotopic non-human mammal model of claim 1, wherein the ovarian tissue comprises a tumor.

14. A method for producing an orthotopic non-human mammal model of ovarian cancer, comprising introducing (i) a first nucleotide sequence encoding an exogenous mutant PIK3CA polypeptide, (ii) a second nucleotide sequence encoding an exogenous mutant KRAS polypeptide, and (iii) at least one inhibitory RNA targeting p53, into an ovarian tissue of a non-human mammal model under conditions effective for the formation ovarian cancer, thereby producing an orthotopic non-human mammal model of ovarian cancer.

15. The method of claim 14, wherein the mammal is porcine.

16. The method of claim 14, wherein the mutant PIK3CA polypeptide comprises an H1047R amino acid substitution.

17. (canceled)

18. The method of claim 14, wherein the mutant KRAS polypeptide comprises a G12V amino acid substitution.

19-31. (canceled)

32. The method of claim 15, wherein expression of p53 is reduced compared to expression in ovarian tissue not introduced with the inhibitory RNA.

33. (canceled)

34. The method of claim 15, wherein ovarian cancer is formed 1-3 months after introducing (i)-(iii).

35. (canceled)

36. The method of claim 15, wherein ovarian cancer comprises tumors comprising diameters for 30-60 mm.

37-41. (canceled)
